(19) United States
(12) Patent Application Publication
(10) Pub. No.: US 2021/0016216 A1
(43) Pub. Date: Jan. 21, 2021

(54) Method and device for holistic protection and virus transmission suppression

(71) Applicants: Victor Popa-Simil, Los Alamos, NM (US); Ioana Livia Popa-Simil, Ann Arbor, MI (US); Andrei Popa-Simil, Albuquerque, NM (US); Liviu Popa-Simil, Los Alamos, NM (US)

(72) Inventors: Victor Popa-Simil, Los Alamos, NM (US); Ioana Livia Popa-Simil, Ann Arbor, MI (US); Andrei Popa-Simil, Albuquerque, NM (US); Liviu Popa-Simil, Los Alamos, NM (US)

(21) Appl. No.: 17/018,177

(22) Filed: Sep. 11, 2020

(51) Int. Cl. B01D 46/44; B60H 1/00; C12M 1/12

(52) U.S. Cl. CPC B01D 46/44; C12M 37/00; B60H 1/00

(57) ABSTRACT

COVID-19 is a large virus with remarkable resilience, propagation and multiplication features that require complex technologic systems and manners to assure a reasonable protection. A transparent, multiuse face mask with valves and for inhalation of air from a specialized filter placed in a cleaner air space, and an exhalation valve that drives the exhaled air via a tube to a filter bladder placed on wrist and from there to a tube that has the release end near ground, or can be connected to the aspiration tube of the infrastructure, that removes it, preventing recirculation. The general convention that defines the usage method is that the building, buss, and any other enclosure provide clean antiseptic air above and consumer s have to release their exhalations down near floor from where the structure to remove them by a controlled air flow similar to clean rooms.

The main condition is that people to place their exhalation exhaust sterilized and placed near ground in order to increase the probability to inhale clean sterile air at the head level. No filter is perfect, and no sterilization unit, therefore the enclosure has to deal with exhaust gas, and remove into the atmosphere safely. The work to keep people inside safe is done by both the enclosure that may provide vacuum lines to collect the exhaled air, and by the people who will use correctly the infrastructure. The system is designed for smart communities for everybody to wear for about 3 weeks when a covid-19 or other virus infection occurs to suppress its multiplication rate, without perturbation in their activity and then returning to normality.

COVID-19 is a large virus with remarkable resilience, propagation and multiplication features that require complex technologic systems and manners to assure a reasonable protection. A transparent, multiuse face mask with valves and for inhalation of air from a specialized filter placed in a cleaner air space, and an exhalation valve that drives the exhaled air via a tube to a filter bladder placed on wrist and from there to a tube that has the release end near ground, or can be connected to the aspiration tube of the infrastructure, that removes it, preventing recirculation. The general convention that defines the usage method is that the building, buss, and any other enclosure provide clean antiseptic air above and consumer s have to release their exhalations down near floor from where the structure to remove them by a controlled air flow similar to clean rooms. The main condition is that people to place their exhalation exhaust sterilized and placed near ground in order to increase the probability to inhale clean sterile air at the head level. No filter is perfect, and no sterilization unit, therefore the enclosure has to deal with exhaust gas, and remove into the atmosphere safely. The work to keep people inside safe is done by both the enclosure that may provide vacuum lines to collect the exhaled air, and by the people who will use correctly the infrastructure. The system is designed for smart communities for everybody to wear for about 3 weeks when a covid-19 or other virus infection occurs to suppress its multiplication rate, without perturbation in their activity and then returning to normality.

### STATEMENT REGARDING FEDERALLY SPONSORED R&D

This invention was made with NO Government support.

### NAMES OF PARTIES TOA JOINT RESEARCH AGREEMENT

This work was part of research of the mentioned inventors.

### CROSS REFERENCE TO RELATED APPLICATIONS

This Application is a continuation in part of the application U.S. Ser. No. 16/823,242 from Mar. 18, 2020.

### BACKGROUND OF THE INVENTION

### 1. Field of the Invention

The present invention relates to a method and device to allow safe meetings for learning, transportation and talking in comfortable conditions even in the presence of a pandemic with airborne and through-skin transmission capabilities and high communicability. The invention utilizes the corroboration between future building infrastructure and personal protective equipment, and personal behavior meant to protect others in order to self-protect. The leading principle is: Keep germs down or flash them out in order to stay tall, and breathe clean air and be safe and sound.

US become the most infect with COVID-19 country in the world due to criminal incompetence, and adversity for truth, hate for others, and instead to effectively localize and eliminate the virus it chose to open schools, restaurants, bars, business, etc. use ghostly freedom concepts as opposed to this nano-machine called COVID-19, a warfare agent with over 200+ k bio-code instructions, that ignores wishful thinking, therefore they decided to live with COVID-19 for many years to come. Because the virus is so complicated, and associated political pressures, the first vaccines may have important collateral effects (encephalitis; neural transmission, muscles inflammation, impotencies, cognitive difficulties, respiratory issues, etc.) as the battle-scars of the virus left on those who “recovered”, creating a “COV” society, similar to the HIV one, therefore those not affected by virus will understand that in order to stay safe, the teachings of these patent may render the cheapest method and auxiliary devices to keep them safe, being redundant and failure enough tolerant. The CDC indications are flimsy, inappropriate for aerosolized transmission, and simply fact that one uses a mask and stay 2 m (6 ft.) apart, sometimes works, and sometimes doesn't, due to air currents, that most people and scientists fail to understand. When this work is simple the game of luck, because based on probability of having an infected contagious person in a group is driven by probability, that depends of the density of contagious persons at large inside a community. Washing hands is also very sloppy, because hands with the layer of dead skin are the less likely place for virus to propagate inside the body's fluids. The CDC also ignores the fact that when an exhaled contagious plume, released at about 37 C (98 F) enters in contact with other person skin that is at about 30 C (80 F) it condensates on it, and virus that is 100 times smaller than a pore diameter propagates through, entering the vein's blood flow. In this case, a person may have about 10 min. to wash the surface in order to stop virus' diffusion, by modifying local skin's chemistry, but no one knows when. Well washing hands is useful from many points of view, and is a way to prevent indirect contamination of face, nose or mouth thinner skin surfaces but that's it. It is expected that in the name of the ghostly freedom rights, constitution, etc., some of the protection requirements to be breached, but the system still fails safe. It is true that any failed to use module drives to gradual reduction of safety coefficient, but some systems may be failed by mobile user, while others are provided by infrastructure and a criminal cooperation is needed to have them failed simultaneously. The system is not designed for those who disrespect knowledge and intentionally breach the protective systems, by sabotaging themselves, but for those who respect the rules, and in certain circumstances there is difficult to comply in order to acquire full protection, and a reduction in protection factor comes as a consequence, but that is not drastic, and enough reserve remains in the system. In these circumstances the system makes very difficult to lose all protection, being modular and functioning independently with some degree of distributed redundancies. Our approach to make meetings among many people safe is to use advanced technologies in order deal with each aspect of virus propagation, survival, and transmission.

### 2. Description of the Prior Art

There seems to be no specific prior art we may consider as direct reference, but we may consider prior art related to anti-bacteriological protection in high risk research laboratories, with bio-hazard level 4 where autonomous respiratory gas and full body PPE (personal protective equipment) are used to assure an individual protection factor of 99.99%

All the actual solutions that aim to prevent the virus transmission are based on covering the mouth and nose, that makes speaking and person identification difficult.

The presently available suits are difficult to dress on and off, and during this procedure the highest contamination risk is high. Another way to stop contamination from spreading is to use a similar design to anti-bioterrorism units with separate ventilation circuits. In these units, the bio-hazardous air and fluids are eliminated via an incinerator, plasma burner, or chemical scrubber.

The system we propose is a modular open system, and any supplementary required function may be added, or its complexity may be reduced as necessary. At this protection system adding or failing to use correctly various modules will decrease the safety figure by a small amount, being necessary 5-6 simultaneous failures the eliminate the protection.

Comfort of wearing, welfare status of bearer and easiness of usage is another important feature added to continuous awareness on protection factor, or how well the system is used, is another feature inventors had in mind when designed the structure and conceived the operating method.

### BRIEF DESCRIPTION OF THE DRAWINGS

FIG. 1—Student reading in a protected space;

FIG. 2—Top air intake modulus;

FIG. 3—Inhalation air preparation modulus;

FIG. 4—Exhaled air cleaning modulus;

FIG. 5A—Transparent mask with sense valves;

FIG. 5B—Classical mask with valves as a surrogate for complete gear;

FIG. 6—Enclosure with controlled air flow;

FIG. 7—Exhaled air evacuation and sterilization system;

FIG. 8—Student walking on a protected hallway;

FIG. 9—Students inside a protected school bus;

FIG. 10—School bus driver and kids walking on street;

FIG. 11—Student working on computer inside a protected space;

FIG. 12—People in a protected bus, train, airplane, meeting room or amphitheater;

FIG. 13—Students inside a protected classroom;

FIG. 14—Detailed chart on aerosols transmission through masks.

### FIGURES DETAILS

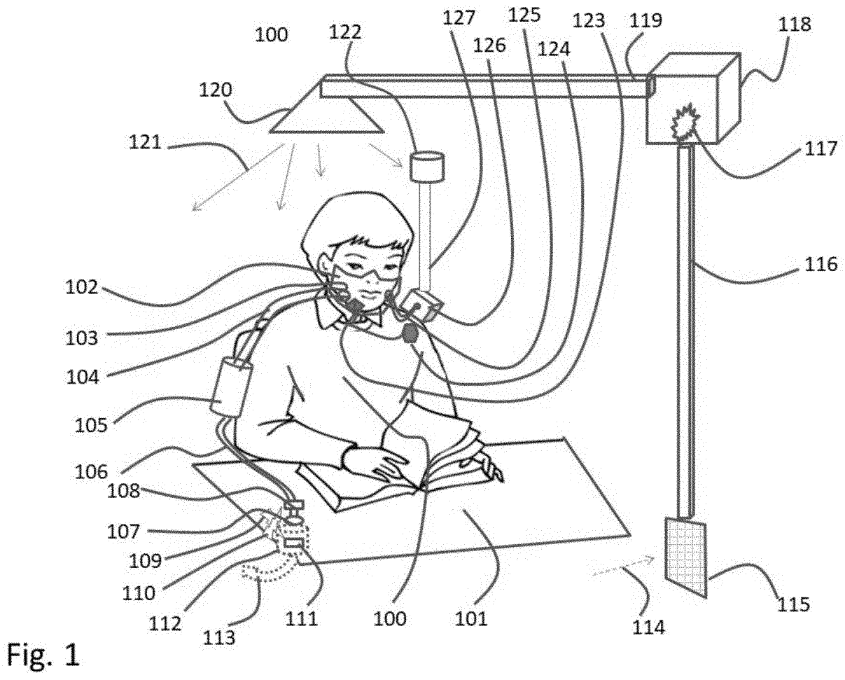

FIG. 1—Schematic view of a student reading in a protected space;

100—Student sitting;

101—Desk;

102—Transparent face mask;

103—Exhalation gas one-way valve;

104—Exhalation gas tube;

105—Exhalation air cleaner modulus;

106—Exhalation gas exhaust tube;

107—Exhalation tube fast connector to drain tube;

108—RFID label;

109—EM communication between RFIDs;

110—RFID reader interrogation signal;

111—RFID reader;

112—Drain tube port with shutter;

113—Clean exhaled air into drain tube;

114—Floor circulating air to HVAC return;

115—HVAC return vent;

116—In wall HVAC return duct;

117—UV-C sterilizer inside HVAC return duct;

118—HVAC unit;

119—HVAC conditioned air duct;

120—HVAC ceiling diffuser inside the room;

121—Clean air from HVAC, flowing downwards;

122—Top air intake modulus;

123—Microphone, thermometer;

124—Electronics module, with loudspeaker, Wi-Fi, display;

125—Inhale air tube connected to one-way valve;

126—Inhalation air conditioning module;

127—Intake air tube;

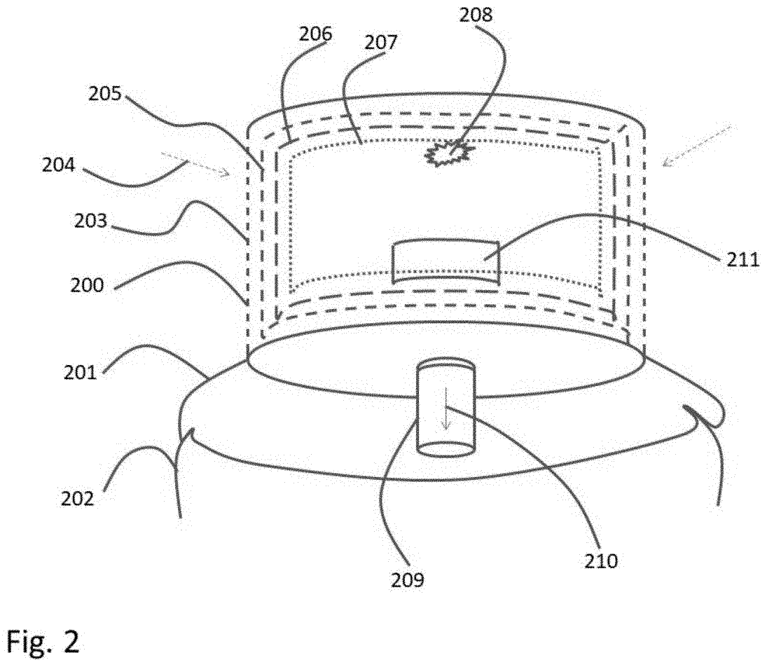

FIG. 2—Top air intake modulus.

200—Primary solid particulate pre-filter protection grid;

201—Support for head;

202—Ribbons;

203—Coarse hydrophobic filter;

204—Air flow coming into filters;

205—Chemical and sterilization filter;

206—Hydrophilic filter-desiccator;

207—UV reflective chamber with bio-active wall;

208—UV-C light source;

209—Connection tube;

210—Airflow direction;

211—Fan and batteries with electronic control;

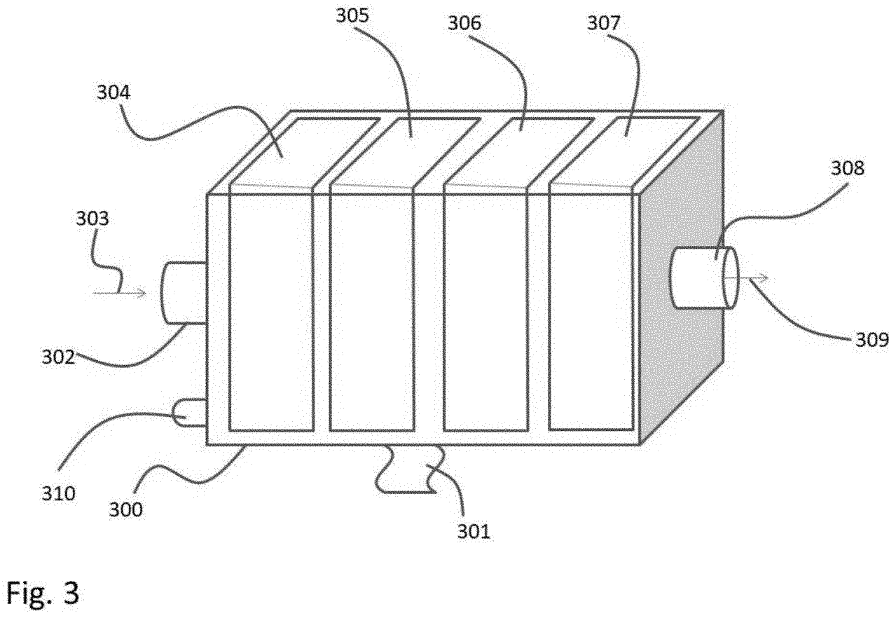

FIG. 3 shows an inhalation air preparation modulus made of:

300—Case;

301—Ribbons to strap on shoulder;

302—Air intake tube;

303—Airflow direction;

304—Intake HEPA filter;

305—Carbon Dioxide stopper;

306—Air preparation filter with medicine applicator for nasal inhalers;

307—Ultrasonic water nebulizer, humidity control, fan and batteries;

308—Exhaust tube;

309—Airflow direction;

310—Oxygen or compresses air input.

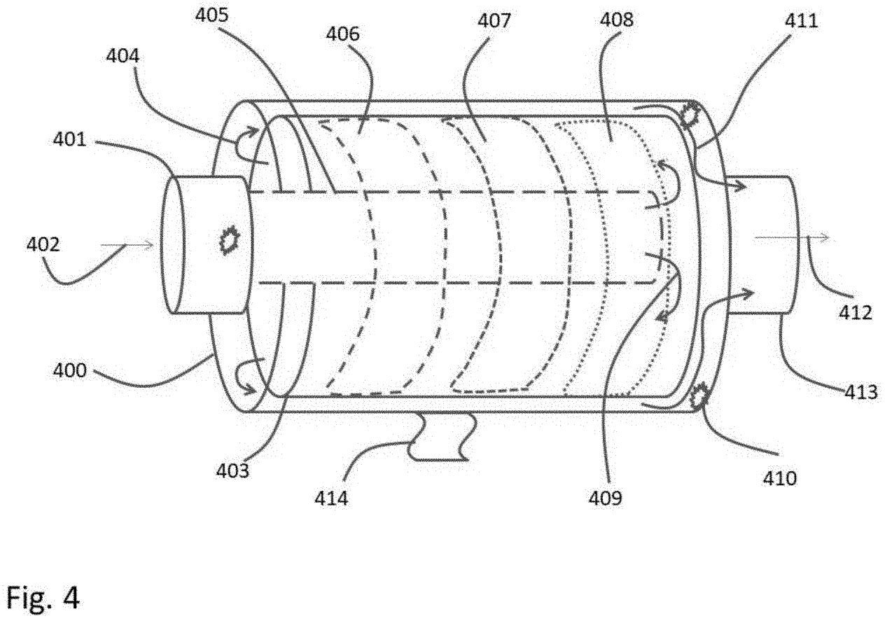

FIG. 4 details on exhaled air cleaning modulus:

400—Metallic case;

401—Exhaled air intake tube

402—Airflow direction;

403—Inner case to cool the air in a heat exchanger structure;

404—Airflow direction in heat exchanger;

405—Central tube to drive air in opposite direction;

406—Desiccator and hydrophilic filter;

407—Chemical and anti-bacteriological filter;

408—Supplementary active charcoal filter;

409—Airflow exiting central tube;

410—UV-C light sources;

411—Airflow exiting the heat exchanger;

412—Airflow direction;

413—Air exit tube;

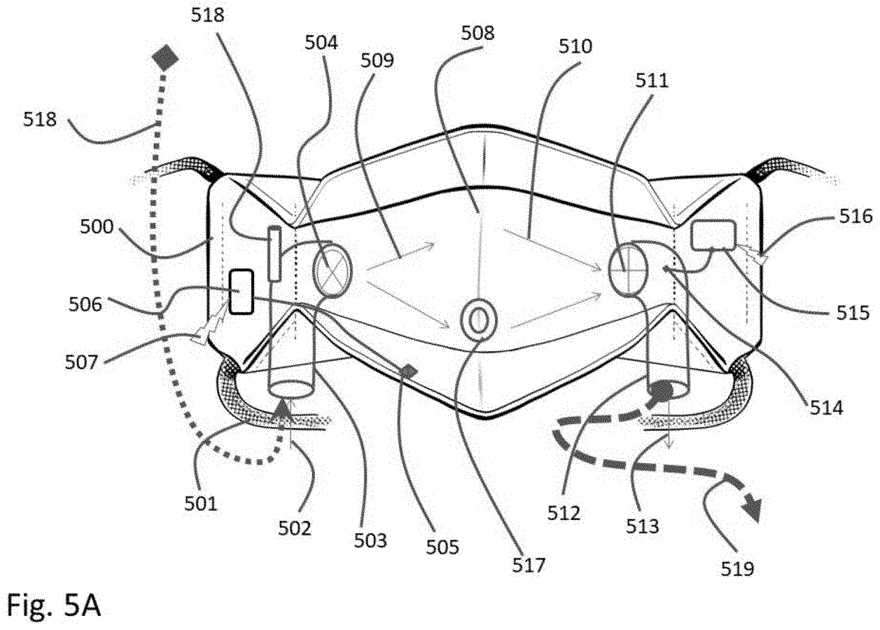

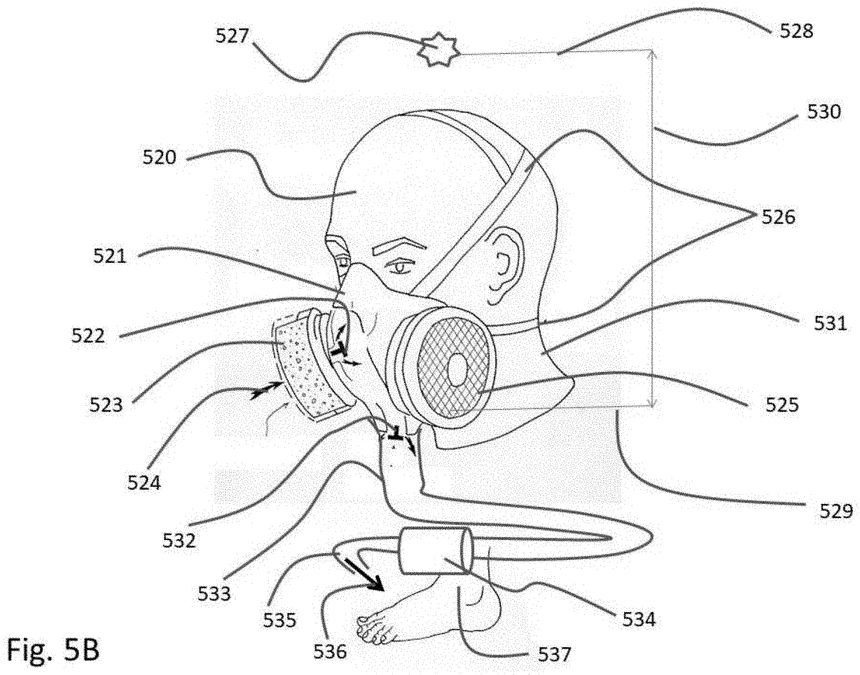

FIG. 5 describes a transparent mask with sense valves

500—Transparent mask;

501—Straps;

502—Air intake sense;

503—Connection tube;

504—One-way air valve;

505—Microphone, thermometer;

506—Electronics box—Wi-Fi;

507—Wi-Fi signal;

508—Front of mask visualization;

509—Inhaling airflow;

510—Exhaling airflow;

511—One-way valve for exhalation;

512—Exhalation tube;

513—Exhaled airflow in exhaust tube;

514—Exhalation sensors (temperature; airflow speed, humidity, CO₂, etc.);

515—Electronics box, with display;

516—Wi-Fi signal;

517—Straw hole with lid for liquid sipping via a straw;

518—Tube for air to protective glasses.

518—Airflow coming from above the head during inhaling;

519—Ari flow sent downwards during exhaling;

FIG. 5B—Classical mask with valves as a surrogate for a complete gear, with necessary improvement as an embodiment of the current patent:

520—Head;

521—Gas mask;

522—Inlet one-way valve for inhaling;

523—Filtering cartridge;

524—Airflow intake;

525—Second filtering cartridge;

526—Head bands;

527—Recommended air intake position;

528—Above air intake upper-level;

529—Lower cartridge air intake level;

530—Difference in intake level;

531—Neck;

532—Exhaling valve;

533—Exhaling tube;

534—Exhaled air sterilization and cooling modulus;

535—Sterilized exhaust air tube;

536—Airflow direction;

537—Ankle level for air exhaust;

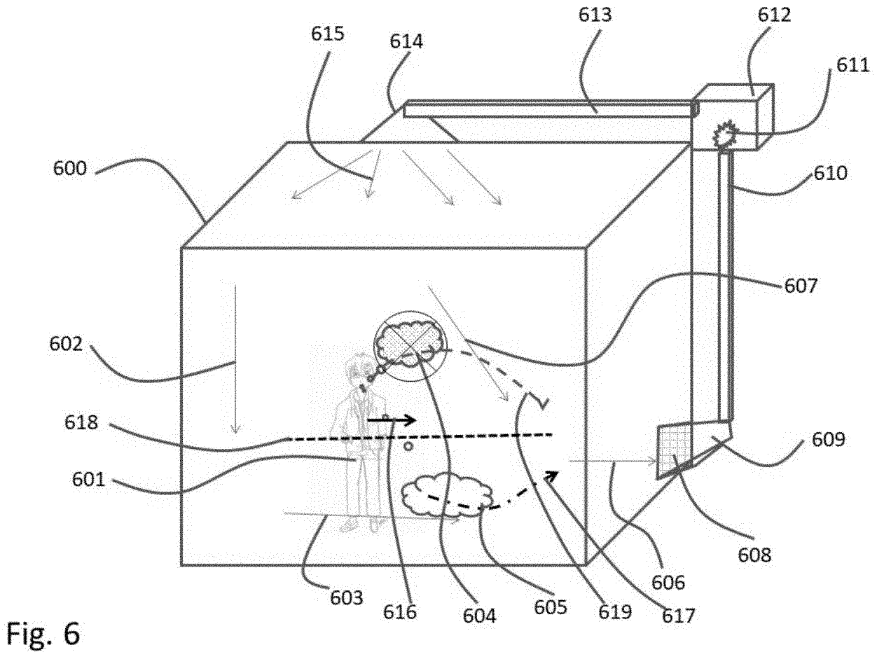

FIG. 6 gives details on an enclosure with controlled air flow,

600—Enclosures walls or airspace boundaries;

601—Person standing;

602—Down going airflow;

603—Parallel with floor air-stream;

604—Exhalation plume without guiding at mouth level, prohibited;

605—Exhausted down exhalation plume;

606—Airflow towards HVAC return duct;

607—Airstream aiming downwards;

608—Return vent HVAC;

609—Inner tube adaptor HVAC;

610—Return tube HVAC;

611—UV-C light source;

612—HVAC unit;

613—Conditioned air exhaust HVAC;

614—Conditioned air distributed funnel;

615—Clean, conditioned airflow;

616—Radius of exclusion around person;

617—Trajectory of the downwards guided exhalation plume;

618—Minimum level of inhalation;

619—Trajectory of an upwards released exhalation plume.

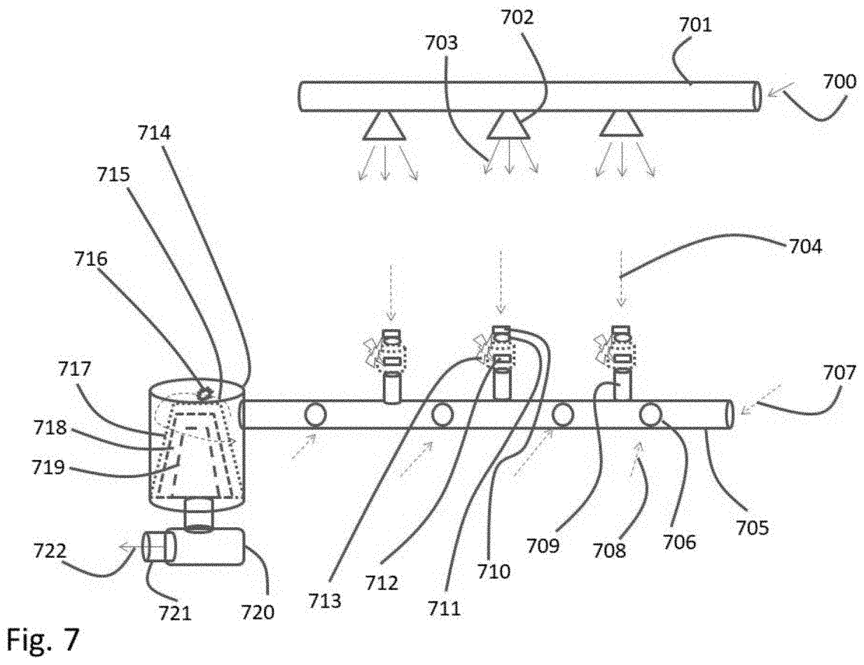

FIG. 7 describes an exhaled air evacuation and sterilization system;

700—Clean air intake;

701—Clean air distribution duct;

702—Clean air diffuser;

703—Clean airflow for breathing;

704—Exhaled airflow collection;

705—Vacuumed drain tube:

706—Floor level collector with lid;

707—End of tube floor level collected airflow;

708—Lateral floor level collected airflow;

709—Person collector exhaled air tube;

710—Person RFID label;

711—Person exhaled air tube connector to drain tube;

712—Drain tube RFID reader;

713—Wi-Fi signals;

714—Drain air collector sterilization system;

715—Cyclone separator;

716—UV-C light source;

717—Pre-filter;

718—Chemical anti-bacteriological filter;

719—Desiccator and HEPA filter;

720—Air turbine;

721—Exhaust tube;

722—Exhaust airflow.

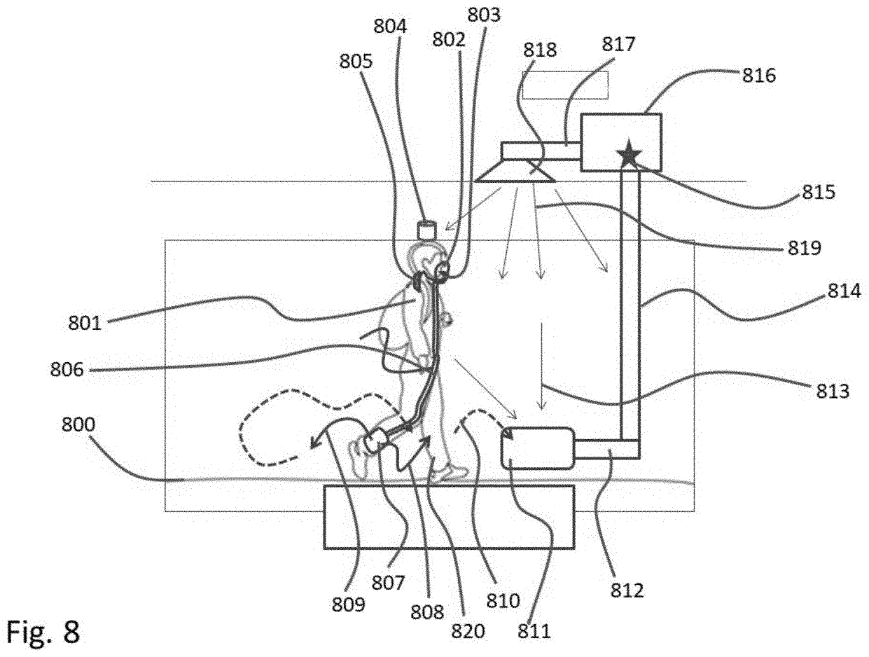

FIG. 8 describes a student walking on a protected hallway where:

800—Hallway's floor;

801—Student walking;

802—Transparent mask;

803—Microphone and other transducers;

804—Upper air intake modulus;

805—Loudspeaker and electronics box;

806—Exhaled air tube;

807—Exhaled air sterilization modulus;

808—Sterilized exhaled air flow;

809—Sterilized exhaled air plume;

810—Airflow near floor;

811—HVAC return vent;

812—HVAC horizontal return duct;

814—HVAC vertical return duct;

815—UV-C light source;

816—HVAC unit;

817—HVAC conditioned air ceiling duct;

818—Conditioned air ceiling diffuser;

819—Conditioned air down flow;

820—Pants and socks;

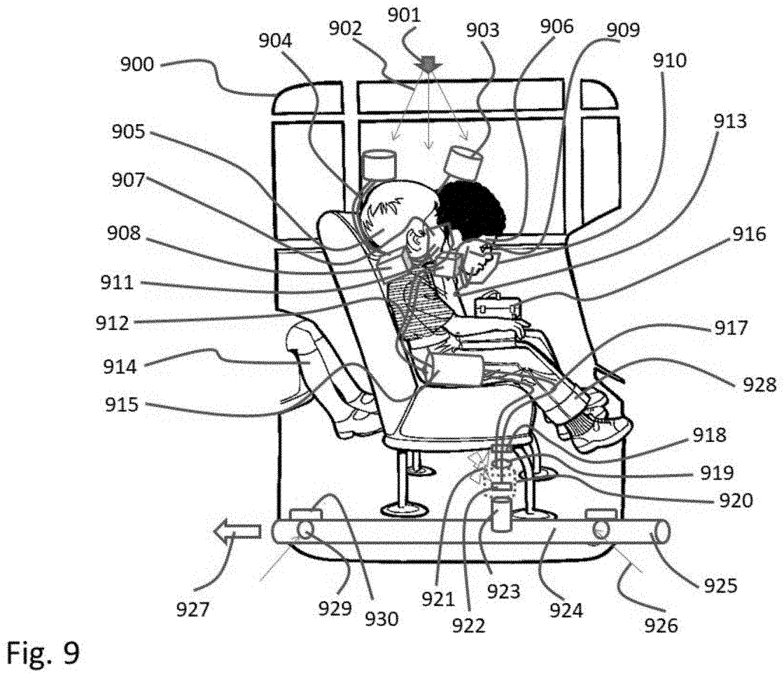

FIG. 9 shows students traveling inside a protected school bus:

900—School bus sector;

901—Clean air input;

902—Clean air flow;

903—Upper air inhalation module;

904—Connecting tube;

905—Student on the aisle;

906—Student at the window;

907—Transparent face mask;

908—Inhalation air secondary filter module on student on aisle;

909—Inhalation air secondary filter module on student at window;

910—Transparent face mask at student at the window;

911—Inhaling air tube;

912—Exhaling air tube;

913—Inhaling air tube at student at the window;

914—Girl student wearing long socks;

915—Exhaling air sterilizer modulus;

916—Student at the window backpack;

917—Sterilized exhaled air exhaust tube;

918—RFID label;

919—Sterilized exhaled air tube connector;

920—Tube connector box to drain with lid;

921—Wi-Fi signal;

922—RFID reader;

923—Connecting tube to the drain tube;

924—Drain tube;

925—Drain tube end lid;

926—Ari flow collection on the floor;

927—Drain airflow to exhaust system;

928—Pants with socks;

929—Lateral lid on vacuumed drain tube;

930—Automatic shutter/opener at presence detection.

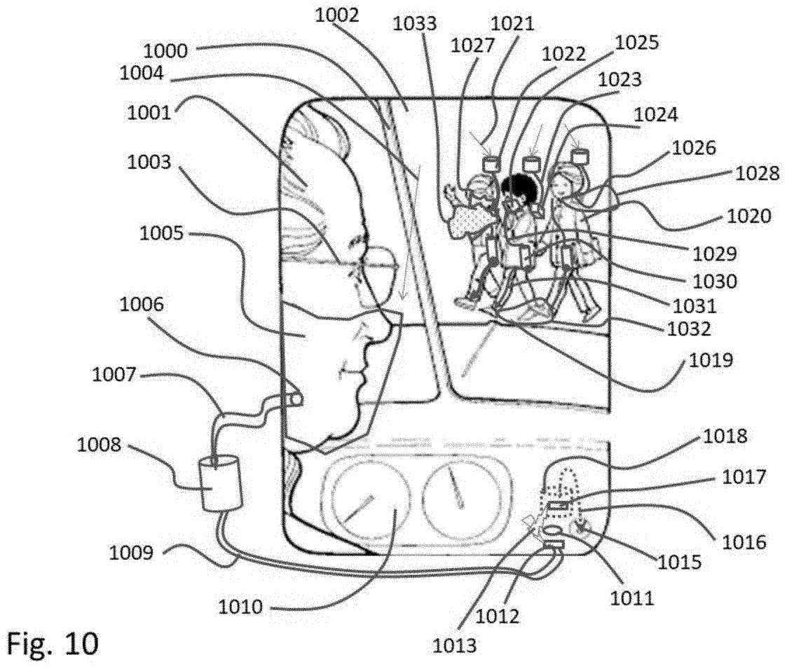

FIG. 10—shows a school bus driver watching kids walking on street;

1000—Front school bus windshield middle ridge;

1001—School bus driver;

1002—Windshield;

1003—Eye glasses;

1004—Air flow in driver's space;

1005—Transparent face mask;

1006—Exhaling tube connector at mask one-way valve;

1007—Exhaling tube;

1008—Exhaled air sterilization modulus;

1009—Sterilized exhaled air connection tube;

1010—Bus dashboard;

1011—Sterilized exhaled air connector to drain tube;

1012—RFID label;

1013—Wi-Fi signal;

1015—Airflow to drain tube;

1016—Airflow direction;

1017—RFID reader;

1018—Drain tube connector and lid;

1019—Sterilized exhaust plume released on the street level by middle girl;

1020—Left side girl;

1021—Air flow inhaling intake direction;

1022—Air inhaling modulus;

1023—Connecting tube;

1024—Inhaling air filtration unit;

1025—Transparent mask;

1026—Tube not connected to mask;

1027—Face mask;

1028—Plume of exhaled gas;

1029—Tube connecting transparent mask to sterilization modulus;

1030—Sterilization of exhaled air modulus;

1031—Tube exiting the sterilized exhaled air at street level;

1032—Plume of sterilized exhaled air released at street level;

1033—Plume of exhaled air released at mouth level through mask.

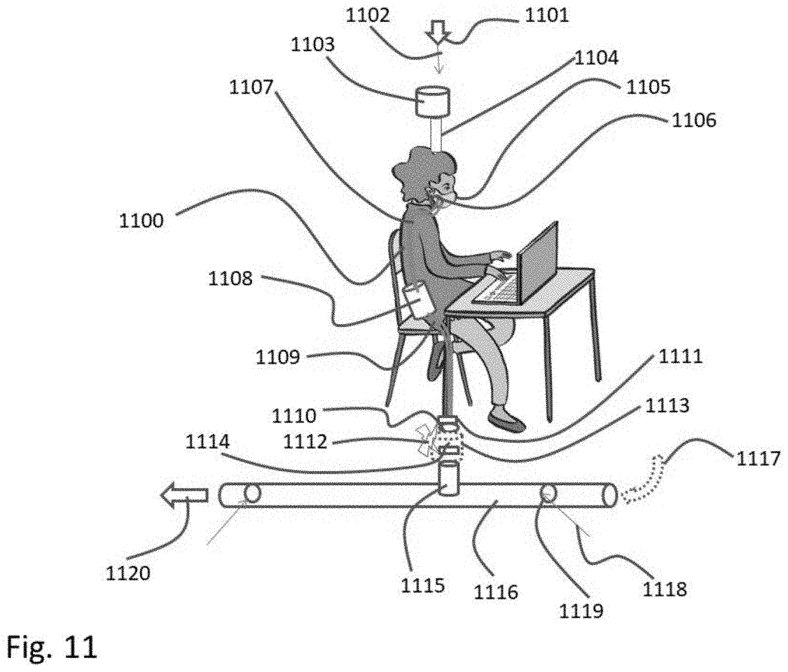

FIG. 11—Student working on computer inside a protected space:

1100—Student working at computer in a protected environment;

1101—Fresh, clean air coming from above vent;

1102—Clean airflow direction;

1103—Inhalation top modulus;

1104—Tube for connection;

1105—Face mask air input;

1106—Face mask exhaled air exit connector;

1107—Exhaled air connection tube;

1108—Exhaled air sterilizer modulus;

1109—Sterilized exhaled air exhaust tube;

1110—Connection of exit tube to drain pipe;

1111—RFID label;

1112—Wi-Fi signal;

1113—Connection box with lid;

1114—RFID reader;

1115—Tube to drain tube;

1116—Vacuumed drain tube;

1117—End of drain tube collected airflow direction;

1118—Lateral drain tube opening airflow direction;

1119—Lateral opening with lid of drain tube;

1120—Drain tube airflow direction to sterilization unit.

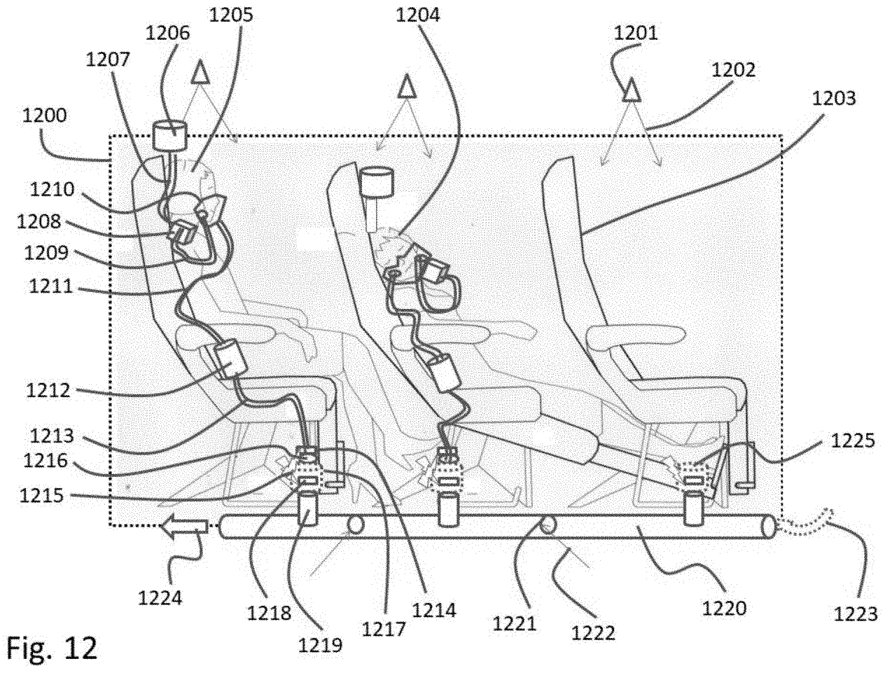

FIG. 12—People in a protected bus, train, airplane, meeting room or amphitheater:

1200—Bus, train, plane, conference hall frame;

1201—Upper vent for introducing clean air;

1202—Clean airflow direction;

1203—Seat;

1204—Person sleeping;

1205—Person seating;

1206—Upper air inhalation modulus;

1207—Upper modulus connection tube;

1208—Respiratory air filter modulus;

1209—Connection tube to mask;

1210—Transparent mask;

1211—Exhaled air connection tube from mask exhaust valve to sterilization modulus;

1212—Exhaled air sterilization modulus;

1213—Sterilization modulus exhaust tube;

1214—RFID label;

1215—Wi-Fi signals;

1216—Connection port;

1217—Connection tube with lid;

1218—RFID reader;

1219—Seat connection tube on drain tube;

1220—Vacuumed drain tube;

1221—Lateral port with lid;

1222—Collected airflow direction;

1223—End of drain tube airflow direction;

1224—Drain tube airflow to sterilization unit;

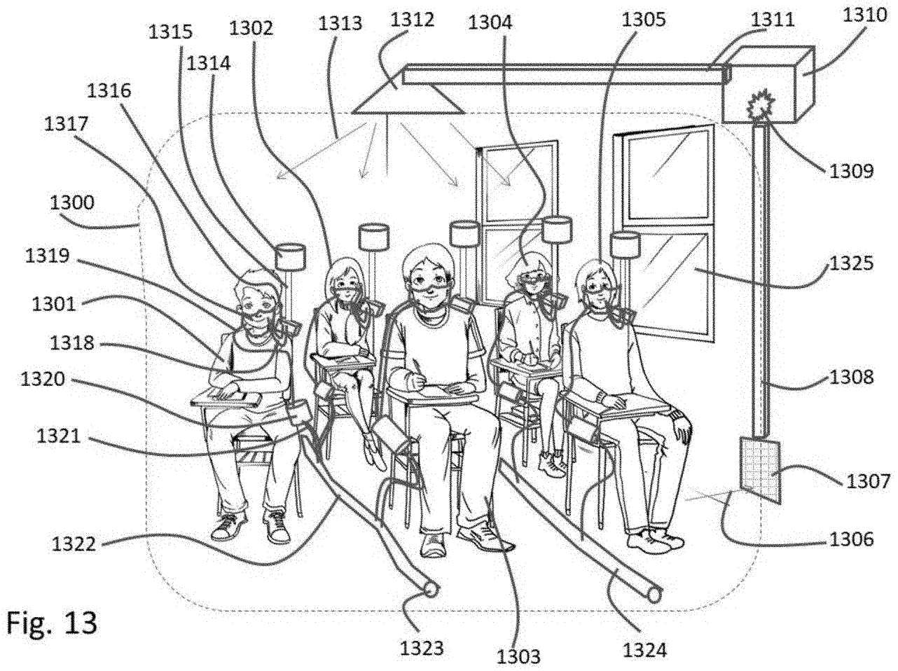

FIG. 13 shows students inside a protected classroom:

1300—Classroom selection;

1301—Right side student;

1302—Middle row back student;

1303—Middle row front student;

1304—Left row back student;

1305—Left row front student;

1306—Air flow direction in the HVAC return;

1307—HVAC return vent;

1308—HVAC vertical duct;

1309—UV-C light source;

1310—HVAC unit;

1311—HVAC horizontal ceiling duct;

1312—Ceiling diffuser for HVAC;

1313—Conditioned airflow direction;

1314—Upper inhalation modulus;

1315—Connection tube to inhalation filter modulus;

1316—Inhalation filter modulus;

1317—Transparent mask;

1318—Connection tube to transparent mask intake one-way valve;

1319—Exhalation connection tube;

1320—Sterilization of exhaled air modulus;

1321—Connection tube carrying sterilized exhaled air into drain tube;

1322—Vacuumed drain tube;

1323—Left side drain tube end;

1324—Right side drain tube end.

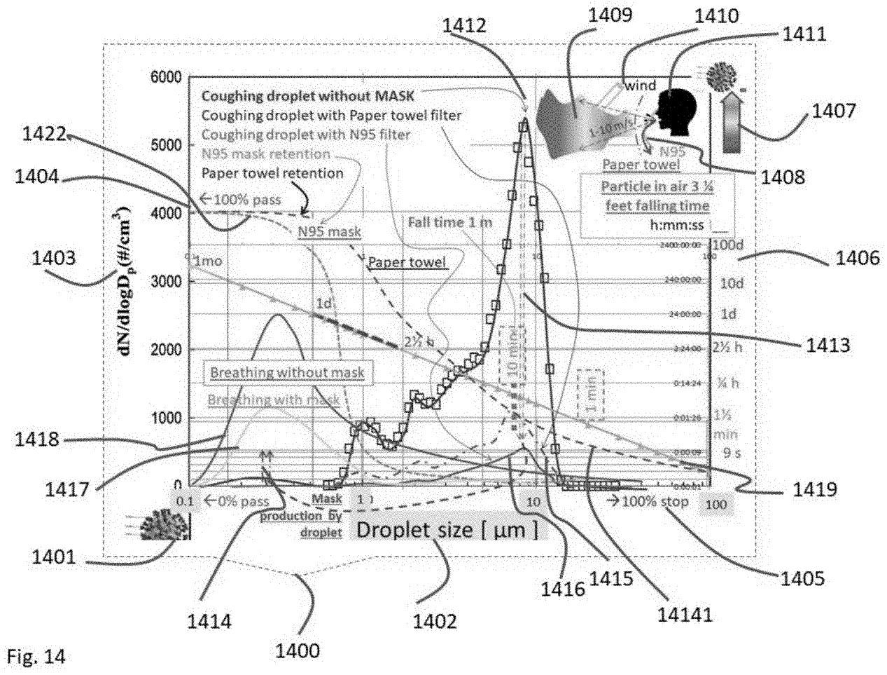

FIG. 14—A chart showing the exhaled water droplets distribution, vs. filter retention capabilities.

1401—COVID-19 average dimension of 100 nm;

1402—Droplet size scale from 0.1-100 microns;

1403—Number of particles per cc as function of particle magnitude;

1404—Particle filter 100% pass upper limit;

1405—Particle filter 0% pass=100% stopping power, lower limit;

1406—Time airborne droplets with 1.5 g/cc density float in air;

1407—Concentration color code for virus inside an air exhalation;

1408—Exhaled air-jet change of direction due to mouth cover;

1409—Exhalation plume without any face cover;

1410—Wind direction;

1411—Head of a contagious person;

1412—Dimensional distribution of airborne droplets while coughing openly;

1413—Retention in a new N95 mask, first shout;

1414—Conversion of large droplets into small droplets released by mask by atomization;

1415—Dimensional distribution of droplets released through a paper-towel filter;

1416—Release of droplets spectrum through an N95 mask (without exhaust valve);

1417—Normal breathing and air exhalation through a mask;

1418—Dimensional distribution of droplets during an open exhalation;

1419—The time it takes a droplet to free-fall 1 m (3.3 ft) based on Stokes force;

1421—Paper towel filter retention curve;

1422—N95 filter retention curve

### DETAILED DESCRIPTION OF THE INVENTION

The inventors consider the developments in siting in class, on a bus, train, airplane and on hallways in a safe manner, in the conditions of stealth propagation of virus using asymptomatic carriers that render the contagious person innocent and unaware of being infected.

These conditions are prominent zones for the spread of contagious diseases and present a risk factor for those around them. As such, technologies as making a mask that has to be easy wearable, having two valves, one for air intake via a tube from a remote filter equipped with an fan to suppress part of the dynamic pressure drop on filter, and another to drive exhaled air through a tube into a retention filter that to stop the water droplets with whatever they contain, and release cleaned scent-free air via another tube which places the exhausted gas near ground.

In order to provide a safe, pleasant encounter, the invention uses high levels of technology to increase the safety and to cancel any viral or bacterial transmission. It starts from real encounters, in schools and working cubicles where due to airborne droplets, transmission of COVID-19 can be spread easily, making people sick with a distribution and intensity that puzzled scientists.

The basics start with the curves of airborne droplets emission of exhaled air from lungs in various regimes, shown in FIG. 14, and their propagation range in open air, with understanding the properties of masks and their operation with liquid effluents, and aerodynamic variations.

It is known that the average adult inhales and exhales around 7 to 8 liters of air per minute when in a rested state. When coughing, or shouting, or exhaling air faster, watery droplets with dimensions up to 20 microns are removed with air, with a maximum of the dimensional distribution of about 8 microns which may travel up to 2 meters in air, accompanied by submicron particles too. When breathing normally or speaking, a person releases mainly submicron watery particles, with a maximum diameter of about 0.6 microns. These particles remain suspended in air for varying degrees of time depending on their size. It is about a week for 0.3 μm particles, to about 1 day for 0.6 μm, and about 2 h for 3 μm particles, being mainly airborne, while for 30 μm particles, it takes about 1 min. to fall 1 meter. After this time in the air, the particles are deposited and coat the surfaces underneath, or those which they come in contact with. Being airborne, these particulates are usually transported in ventilation systems, passing through AC systems and spreading through the entire building. When a sick person is inside, and it is assumed of being contagious with COVID, viruses released will be included in exhalation droplets mainly. It is unclear how many contagious agents per droplet, because that depends on the contagiousness level. On average, an exhalation contains about 10,000 droplets/cc. When the AC system is set on cooling, the virus will concentrate in condensing water drain, and in part will pass unaffected, but when it is hot, some of the viruses will be damaged by the heat, but not all of them, and the virus survival ratio will vary from case to case.

In order to overcome these technologic complications, the system uses the lower exhaust tube to connect it in the enclosure vacuumed exhaust drain, where only the exhaled air is collected and sanitized, then, released in atmosphere. The system formed by a transparent mask, a polyethylene sheet with two valves and flexible tubes connectors, with no filtering capability, by itself is adding a protection factor of 20 dB, by reducing the contamination space from 100 cuft down to about 1 cuft (1 cuft=28.317 liter), but the exhaled air is in the direct reach of noses of others who may inhale it. Using the exhalation flexible tube and placing the exhaled air at floor level, brings another, 10 dB attenuation, because when the exhaled air was released surpassingly contained 10,000 droplets/cc, but when by buoyancy difference and diffusion rises back at others mouth level it is already diluted, at levels less than 1,000 droplets/cc. If the flexible tube is metallic, and acts as a heat exchanger, it dims the exhaled air buoyancy making it unable to rise back to mouth level immediately, and until it reaches that level suffers a dilution down to about 100 droplets/cc.

Like it or not, if this happens into an enclosure, the exhaled air accumulates, and in time, it rises the danger level or risk factor. Basically, after 1 hour the safety factor is reduced by 15 dB. It comes now the role of the enclosure to do something with the accumulated exhaled air. The actual buildings are mainly recirculating it through the HVAC systems, and in cooling mode, in condenser a part of droplets are removed as condensate, in a tank or directly outside via condenses drain tube, that brings a reduction of 3-6 dB of the risk factor, but what remains is mixed in the enclosure, which gives a 6-10 dB risk reduction by dilution. A room recirculation of the entire volume is by design to be 4-10 h, and at each this interval a 3-6 dB is added at protection factor. Adding an UV-C sterilization light source, with a spectral UV power of 3 W/1 ton of ice/day (12,000 BTU/h) is praised to kill more than 90% of germs adding a 10-20 dB at protection factor. An even better solution proposed by the actual patent is to have the return of the HVAC at floor level, and that will create a down-flow current in the room, adding a 10-20 dB at protection factor reducing the diffusion upwards, and guiding all airborne droplets through HVAC. Adding the UV-C light source inside makes the building system by itself provide a protection of 30 dB. A condition is that the fan works continuously when there is presence in the building.

In the case a person is mobile, walking on hallways, or in a room, the enclosure air conditioning system has to introduce clean air above, that to flow down, similar to clean rooms and be collected by the HVAC air return near floor, recirculated through filters and UV-C light and reintroduced via AC appropriate heat exchangers. The technologic system has to be designed to be aware of this restriction and manage it. Based on the explanations above this system is providing 40-50 dB protection factors without using any supplementary filters.

The failures in this system are produced by user, releasing exhalations at mouth level, and by ventilation system due to drafts created by door opening to a windy outside or other pressure room, or by HVAC operation with fan on automatic mode, and during that interval of time the fan to be shutdown, HVAC being on stand-by. No mask by a contagious person and no fan running make the risk factor high and protection is down to 0 dB. In order to have some protection, we have to introduce individual filtration modules, as a personal and society protection feature. As it was shown in FIG. 14, added as a courtesy to the reader, an N95 filter is producing a 12 dB attenuation and a N-100 about 16 dB when is well made. It has to be mentioned here that any material layer, introduced in front of mouth-nose, that stops the direct airflow from exhalation, and reduces its range from 2 m (6-8 ft.) to ¼ m (1 ft) introduces a community protection factor up to 20 dB, based on the reduction on contaminated surface and volume. The practice shows that this attenuation is not enough and, based on statistics, between 1-4% of first responders got sick using these protections, value resulted from medical studies and statistics too. For example, by April 17^{th}, US registered 807 k cases, from which 9 k were first responders, that being 1.115%, value that is utterly unacceptable for civilized human society, which had the capability to learn and adapt. In China, the coefficient was 4% due to novelty of the virus and first uneducated exposures to it, using normal protective gear. There is a large variety of filters that may be customized on user, but first, one has to find a safe place from where to inhale air, and that is above the head.

When air is inhaled, it has to first be cleansed of solid particulates to prevent clogging of the filters. Then, it has to use the fact that viruses travel inside water droplets, and use a chemical barrier to stop and destroy or debilitate them, such as salty filter. In such a micro-crystalline filter, when an aerosolized watery droplet containing SARS comes into contact with a salt micro-grain it dissolves it, and in that solution, salt ions have direct access to germs chemically interacting with them, modifying their structure and disrupting their functionality. If UV radiation and/or catalysts are present in the process a synergistic environment is created, and germs' damage is amplified. When water evaporates, salt recrystallizes trapping germs inside micron size grains, which even if it becomes lose, it is easily stopped by the next filter. In some cases, removing CO₂ in alkaline filters might be an issue and some odors using a charcoal active filter, then, one has to remove the aerosol particulates released by these filters, and may apply UV to further kill germs and retain. This kind of filter will add a 30 dB protection for inhaled air, but released air is dry. Air qualities have to be restored, and a supplementary filter on shoulder may be recommended which to humidify, scent air and eventually add inhalers, to medically treat persons and add a fan to partially remove the pressure dynamic drop on filters. This will add about 10 dB to protection factor.

As one of the terrible features of this virus is the stealth propagation, being carried by asymptomatic people, or infected contagious people in the preliminary phase when sickness symptoms have not been triggered, it will be good that everyone who wears a mask, to take the exhaled air and sterilize it, this way, assuring that they protect others from their unknown exhalations. A sterilizer modulus may be added, that has a chemical anti-bacteriological filter that destroys the germs, a CO₂ retention filter, and UV-C light source and a desiccator filter followed by a fan and a heat exchanger filter to lower the air temperature to environment's temperature, releasing the air by a flexible tube at feet level. This will add a 30 dB protection.

When stationary, inside an enclosure, the exhaust tube may be connected to a vacuumed drainage system that further sterilizes and disposes it in environment, in a safe manner. This system adds more than 20 dB in protection, and may be as high as 50 dB.

If all the systems are applied simultaneously, that may drive to protection factors over 100 dB, at maximum reaching 200 dB that is near total protection for aerosol transmission.

It was mentioned before, but not stressed enough that this COVID-19 virus propagates through skin too, with a 31 10 dB success factor, but high enough to produce sickness, and when directly exhaled at 100 F (38 C) it condensates on skin surfaces that has lower temperatures down to 80 F (27 C) and people have to cover those surfaces too. Applying this protective system intensively zone wide for 2 months may reduce the contamination rate and eliminate the virus. In this circumstances, the problem moves to controlling the trespassing through borders, test repeatedly and quarantine them, in order to be sure that the normal life is not perturbed by these new cases and the entire region has to return to enhanced protective measures, as described above.

The main embodiments of the inventions are related to the process to stop water droplets from reaching outside, no matter their potential virus load, by using a drain and chemical filter canister. The droplets produced from the customer's mouth and nose may be contagious, containing live bacteria and viruses that may be treated as a bio-hazard and a desiccator may be used to take them out of air. In order to prevent exhaled air that is warmer than the environment to go upwards, it is cooled down in the canister and then, released at the shoe level.

As the mask has a controlled input from a filter placed usually upwards, it may also include a nebulizer that to produce humid, salty or scented air, being useful for asthma, COPD treatment or to improve mood of the bearer.

This system is efficient in stopping flu pandemics also, in spite the virus size is much smaller, at about 40 nm, the protection factor will be smaller, but enough for transmission suppression.

### 2. Best Mode of the Invention

FIG. 8 and FIG. 13 show the devices in the best mode contemplated by the inventors where the students and enclosures protective systems work simultaneously to provide the best protection possible. Some bio-medical parameters as temperature, humidity, breathing rate and flow, air pressures, and eventually pulse rate, and blood oxygen may be used. This data acquisition system may be connected to instrumentation that make selective communication possible and internet connection that are embedded in the present invention.

The whole goal of the system is to prevent transmission of virus, even if one has a contagious person in close proximity but that is not enough and testing will be required to identify and treat the asymptomatic carriers, while the system data acquisition helps in identifying in advance any modifications in body's welfare and parameters that may not be the effect of COVID-19 contamination, only but a large range of other germs and preexisting conditions.

The invention corrects previous deficiencies of the previous method, as follows:

It improves the safety of each person by separating the air spaces and destroying all organic matter by a combination of technologies and a redundant number of layers, which finally collects all fluid effluents emitted by a person and sterilizes them continuously maintaining the person in a fresh, safe environment, and cleaning even more in-depth the environment for the next person.

As one can see, modularity is an important feature for maintenance point of view, each module has to be connected to air intake and outtake ducts, for a full protection.

Best application of the invention is explained in FIG. 8 and FIG. 13, but it is not limited to the specific application presented and there are also some applications that do not require such complex equipment. A simplified version is possible to be used and gradually upgraded. It may start with versions that make breathing flushing out, add UV light sterilization, but transition time of a virus and probability of damaging a virus by this method greatly remains unknown and protection failures are probable. The second stage in upgrading the structure is to apply an intake air filtration unit on student, that to assure a decent protection even inside a contaminated airspace. But without extraction of the contaminated air, it will remain in the location, therefore one may take fresh air from outside, as fresh as that one may believe the air is outside and introduced by a fan, make it flow down and flush it back outside in the air. The problem is that temperature and humidity of air outside varies with weather, and one may need to control air parameters, therefore an AC unit introduction may be advised. In order to make it safe, one has to destroy the effluents, but it must first be collected or the entire mass of air involved in the process must be sterilized. Collection in desiccators and/or scrubbers is useful, sometimes unavoidable when trying to use same air.

When wearing many filters in series, their dynamic pressure drop add, reducing the pressure at mouth level during inhalation as well as when exhaling the pressure at the mouth level is increased, and the cumulative effect translates in air volume reduction and oxygen depletion or a difficulty of breathing. The dynamic pressure adjustment system, is met to have fans in upper modules and lower sterilization module to work in push-pull mode, such as when person inhales and pressure at mouth level trends to be lower than outside pressure, the intake circuit fans accelerates producing a higher pressure on mouth, then stop, and when person exhales the fan in exhaust circuit accelerates making pressure on mouth negative, and by this procedure the oxygen intake may be improved by 5-10%, making easier to breathe. Additionally, an oxygen port may be added in respiratory air preparation modulus.

### 3. How to Make the Invention

The best way to make the invention is to start with the transparent mask, which is basically a plastic sheet thermo-formed in order to make it acceptable to be easy for wearer, be stiff enough while uses a thin foil, that does not attenuate the sound too much. A main requirement is to minimize the distance between the face and mask in order to minimize the residual air inside the mask, and keeps fresh air weight high, preventing re-breathing. Two one-way valves, one for air inhalation and one for air exhalation are mounted in lateral orifices, inside the mask, on two connecting elbows pipes, with diameter from ½″ up to 1″. If the tubes are narrow, they are easy to wear, but airspeed inside is higher, while if the tubes are large, will be harder to wear, but the dynamic pressure drop inside will be small. Flexible tubes with smooth inside are preferred to rough ones, because we like the flow to be laminar, without wall effects. The first step is to customize the inhalation filters, to match the needs of the carrier. It is preferable that the filter to be placed above the head, as a hat, or on a semi-rigid tube, as presented in the most of the drawings. Inhaling air from higher levels is an advantage, as it is free of larger droplets over 20 microns that have short falling time, but airborne aerosolized droplets first are climbing, being hotter than the environment and later may descend, or being carried by the air currents, and may carry bio-agents, being a result of a contagious exhalation. To deal with that need to introduce filters.

First, one needs a protection against rain; made by a hydrophobic mesh, say a 200-400 mesh that to act as a pre-filter too, or to be followed immediately by a specialized pre-filter. Then, one has to use the advantage of COVID-19 embedded inside water droplets—using a salt impregnated filter or if possible, impregnate the pre-filter in salt. This is effective as water droplets dissolves salt crystals, creating a ionic solution that attacks virus damaging it, and when water evaporates it recrystallizes sealing virus inside a large particulate, that even it is dislocated it's easy to be retained by a HEPA filter. A heap filter is recommended to stop any other small particulate down to 0.3 microns. All this filtering capacity added about 30 dB at safety coefficient, but still 1 in 1000 of viruses may pass through, and dry COVID-19 also passes through. To do a little better, the inner space will be used for UV-C sterilization light source. This will require a battery or electric connection to a battery, consuming about 3 W. If one thinks to use it for 8 h, it will need a 7 Ah, 5 V battery to deliver 24 Wh out of about 35 Wh. This is a ½ lb. battery, to be mounted on the belt.

A tube may connect the filter directly to mask or one may introduce another filtration unit, that will make the air easier to breath by adding some humidity, using an alkaline filter to remove residual CO₂ or the scents with an active charcoal filter. Of course, all added filtration units produce a dynamic pressure drop, and that may compete with capability of human lungs to inhale air that at its maximum is at ¼ bar, the person getting as much air as being at 10,000 ft altitude.

To compensate for this a differential pressure sensor may be added to control a fan that to compensate for the pressure drop, delivering a pressure in the inhalation valve about the same from the open air. That will require battery power too, and that is why the battery is a little bit larger having 35 Wh or more. LiPo batteries will be preferred for this application.

Now, as the person protected him/herself against failure in enclosure system adding about 50 dB in protection figure, it is the time to protect others.

For this, the simple solution is to make each person exhaust be released as low and as far as possible, at the foot level, for convenience. Once the plume is there and may be contagious, infrastructure has to deal with it. The safest system is to deploy a drain tube to collect exhaled air only, to sterilize it and dispose safely outside. A problem remains when the person is mobile and cannot be connected to the drain tube, but still putting his/her plume low. In this case, drain tube has to have the capability of absorbing air nearby and treat. In this case, the volume of air to be treated increases by more than 10 times. The best solution is to make the enclosure's HVAC system have the air input on ceiling and air return near the bottom.

It is an individual's duty to protect their community, and in this manner community protects it, and a supplementary sterilization module may be added on the belt or strapped on the leg, which sterilizes his exhalation and stops the watery droplets and eventually the CO₂ in special cases, cooling down the air at room temperature. The sterilization module may have some pressure compensation, in order to make the person feel as breathing in open air and not through the exhaust system, but this requires the use of battery power, and will increase the weight of the battery and its electric capacity.

The FIGS. 1 and 8-13 show different instances on how the protective equipment described above have to be worn, and common failures and how much in protection coefficient is lost or gained, for each feature. FIGS. 2-7 show each modulus, with a description on what has to be added and what might be the expected effect. The method describes practically a procedure to eliminate COVID contamination by a strong social response where when an infection case is reported the entire zone affected to wear the system of protection and stay healthy, while giving time those asymptomatic carriers to be identified, insulated and treated. The duration of daily use of the equipment may be between 2 weeks to 2 months, without interruption in the process. If collateral measure of backtracking the contagious person's interaction quarantine, test and treat infected persons are taken, there is the risk for those who want to remain uncontaminated, that what was described to become the new standard of living in the presence of contagious people without getting infected. This part was not desired by these patent authors. One of most efficient contamination backtracking detection is to use cell phone GPS and Blue-tooth functions to calculate the distance between persons and duration of an interaction, while for open spaces wind direction has to be consider, when calculated the hazard coefficient of an exposed person.

The risk factor (R_{f}) or protection factor (P_{f}) is defined in dB (deci-bells) where the value is given by formulae: R_{f}=10lgNⁱ/Nₐ; where R_{f} is the risk factor; Nⁱ is the number of aerosols inhaled, and Nₐ is number of aerosols available during the time of interaction calculated by multiplying inhaled volume with time of interaction and the concentration of aerosols droplets estimated as function of interaction distance and filter used.

The evaluation of the needed protection factor is based on the FIG. 14 conclusion that a contagious person talking and breathing normally exhales about 10,000 watery droplets per cubic centimeter, which for a person breathing 1 min, drives to 100,000,000 droplets in about 10 liter of used air, and if highly contagious each droplet contains at least 1 virus entity. In reality this ratio depends on contagiousness level, which is variable, but we considered near maximum. If one breathing through his filtration system and inhales that atmosphere inside the exhaled plume, in order to get sick theoretically, it has to inhale 1 entity, inside 10 liters of inhaled air. To reduce the initial concentration to 1 droplet/cc one has to have a protection factor of −80 dB. At this level corpuscular statistic fluctuations have to be considered where many times there are 10 l volumes with none entity, and sometimes appears a volume with several entities. In these conditions for one person to get sick, several minutes of breathing are needed. In order to not get sick in few hours of staying in contaminated air one needs about −120 dB in protection factor. Reaching this value for a single unit is difficult, it was presented in the patent application U.S. Ser. No. 16/823,242, and is competing with individual compressed air supply, that is complex and difficult to apply for community protection, and that is why the protection was modularized and distributed, making its good operation depending of three independent factors (bearer; drain tube operator; building operator) in order to reduce the probability of a total failure. The 5 modules on bearer (intake modulus, respiratory air modulus, mask, sterilization and exhaust connection) are also autonomous and they signal their operating status keeping bearer and others aware of that in real time. In a good faith operated system the probability of total failure is extremely low.

### DETAILED DESCRIPTION OF THE FIGURES

The technology is intended to prevent the spread of COVID-19 and other viruses while also being as comfortable as possible for users, but most of all be redundant, fail safe and assure a high level of protection. To allow this, a sophisticated air flow control system as well as other virus propagation suppressors clean air generation is required, and the enclosure infrastructure needs to act synergistically with the equipment on the individual users.

FIG. 1 shows a schematic view of a student reading in a protected space, where student, 100, is sitting at a desk, 101, wearing a transparent face mask, 102, that is transparent in order to see his mouth, and made out of low density material, to produce small sound attenuation.

Mask has no filter on it, is just a face cover equipped with an exhalation gas one-way valve, 103, and an inhalation air tube connected to one-way valve, 125, all placed as near as possible and comfortably achievable to the face, as to leave a very small residual air volume between.

The exhalation gas tube, 104, is connected to an exhalation air cleaner and sterilizer modulus, 105, which after cools down the air, releases it via an exhalation gas exhaust tube, 106 into exhalation tube fast connector to drain tube, 107, and from there into a vacuumed drain tube port with shutter, 112, for further dispositioning.

In order to know who is connected and who is not inside an enclosure an RFID label, 108, is placed at each hose terminal, which via EM communication between RFIDs, 109, initiated by a RFID reader interrogation signal, 110, emitted by a RFID reader, 111, that communicates Wi-Fi with a computer detects immediately who is connected and who is not, and triggers the alarm, were those not connected without a good reason, just invoking their constitutional freedoms of infecting others to be eliminated from the enclosure, in order to restore protection factor on maximum possible.

The exhalation air sterilization modulus attempts to clean the exhaled gas of any germs, but in mass production and usage, it is not sure that may always deliver an absolute cleanup and even when it operates correctly, still allows some germs to pass through, therefore clean exhaled air is sent into drain tube, 113, while the leakage and air escaped from mobile users, not connected to the drain tube is trained by, floor circulating air to HVAC return, 114, into HVAC return vent, 115. Inside wall there is a HVAC return duct, 116, that contains an UV-C sterilizer light source, 117, inside HVAC return duct, before HVAC unit, 118, because if it is placed inside

HVAC conditioned air duct, 119, it will be subject to high or low temperatures that may affect its reliability. As usual, an HVAC system contains an air filter, but the quality of air filtration seldom reaches level of MREV14, due to the need to maintain the dynamic pressure drop low, for energy efficiency reasons, and we cannot count on that to reduce germs.

The conditioned air is inserted back in the room via an HVAC ceiling diffuser inside the room, 120, making clean air from HVAC, flowing downwards, 121, and pushing downwards any airborne aerosols. This is good, because for air inhaling a person may set a top air intake modulus, 122, up above the head, sitting on a semi-rigid intake air tube, 127, connected to Inhalation air conditioning modulus, 126, where both have roles in air cleaning for user's protection. This feature is important in environments where the exhalation air drain, and HVAC does not work, or fail but it does not assure an absolute protection. In case air is more infected, advanced protective systems based on catalytic burner and respiratory air restorer, or compressed fresh air tube has to be used. There are difficulties of communication with the mask over the mouth, and that may be improved adding a microphone, thermometer, 123, connected to an electronics module, with loudspeaker, Wi-Fi, display, 124, which transmits voice outside, cancels local noise for Wi-Fi local or remote communication, and may provide supplementary functions as bio-medical parameter measurement and analysis. Using the mask support, one may easily measure the temperature of the exhaled air, may use an anemometric thermometer and measure the flow rate, humidity, and deduce breathing rate, which combined with gyroscope and accelerometers on the phone, may go to metabolic functions monitoring. A microphone may be added on neck as a frugal version of noise reduction, or a microphone array may be used. The local display may show the bio-parameters on the mask, and when something goes red a supervisor may identify that there is a problem. A pressure sensor in the mask is connected to upstream and downstream fans which can adjust the dynamic pressure such as when inhaling air the pressure at mouth level to be higher, and when exhaling the pressure to be lower, also allowing some tangential flow such as the residual exhaled air to be washed down.

FIG. 2 details top air intake modulus that plays an important role in individual protection. First, it is placed on top, as high as possible, because supposing that exhalation is driven down, even by diffusion the germs that will reach that level will be diluted at least 10 times, so a 10 dB in average may be added to protection factor.

The modulus has at exterior a primary solid particulate pre-filter protection grid, 200, and in some cases when is mounted instead of a hat it has a support for head, 201, with ribbons, 202, to buckle up on the head tight. This exterior shell has to be a rain protection also.

A coarse hydrophobic filter, 203, is needed on rain time to reject water droplets and keep inside as dry as possible, stopping larger droplets from air flow coming into filters, 204, such as to maintain chemical and sterilization filter, 205, dry, able to kill germs, bacteria and viruses.

Because most of the aerosolized viruses travel in watery droplets, when such a droplet touches a salt grain, impregnated on a filter, it dissolves that grain totally or in part releasing the ions in solution that chemically reacts with virus, damaging it. When water evaporates the salt re-crystalizes catching and sealing inside the left over from the virus. Even if the salt grain is dislocated from the support and flies with airflow, it has a considerable dimension easy to be stopped by a HEPA filter after that. Using UV in this environment the 3 eV photons act as a catalyst for chemical reactions which destroys the virus by increasing ionization level and molecular separations.

A Hydrophilic filter desiccator, 206, is added to stop the aerosolized droplets that successfully passed through the chemical, anti-biological filter, stopping the water they travel with.

This chain of filters may not kill all viruses, therefore an UV reflective chamber with bio-active wall, 207, is built in center where an UV-C light source, 208, is applied killing a part of the survival viruses before reaching the connection tube, 209, where the UV-C light extends inside the tube following airflow direction, 210, in order to maximize the exposure time.

Up to now, the protection factor may be found adding the protections induced by each element of the modulus as shown in Table 1:

| Element | Min. P_{f} | Max P_{f} | Comment |
|---|---|---|---|
| Top position no | −3 dB | −10 dB | Air accumulation |
| enclosure air control | | | may worsen the safety |
| Top position | −6 dB | −20 dB | The element is sensitive |
| enclosure | | | to HVAC mode, |
| with HVAC | | | and UV-C may add 10-20 dB |
| Top position enclosure | −20 dB | −40 dB | Is sensitive to HVAC |
| with HVAC and drain | | | mode and tube |
| | | | connections to drain |
| Pre-filter | −3 dB | −6 dB | Is designed to stop |
| | | | large particulates and |
| | | | repel rain droplets |
| Chemical anti- | −10 dB | −20 dB | Has limited usage time |
| bacteriological | | | in the range of 1-2 |
| filter | | | weeks and needs replacement |
| Hydrophilic- | −6 dB | −12 dB | Stops water vapors |
| desiccator | | | drying the air |
| filter | | | |
| UV-C light source | −10 dB | −20 dB | Depends of UV power |
| | | | and airflow speed |
| Total P_{f} | −32 dB | −98 dB | In practice one may |
| | | | sure count on −20 dB |
| | | | to −50 dB as multi-parameter |
| | | | function. |

The protection factor is better than the actual N100 filters that deliver about −25 dB

Additionally, a fan and batteries with electronic control 211 may be added, that will work synchronous with inhaling phase, reducing the pressure in the mask.

FIG. 3 shows an inhalation air preparation modulus that is supplementary to the first filtration modulus, and has the role to prepare a nice respiratory air, add humidity, inhalers, scents such as to be comfortable to breathe, and wear the mask.

A case, 300, ribbons to strap on shoulder, 301 is customized, having a semi-rigid air intake tube, 302, holding up the air input modulus, or a fully flexible tube when the air input modulus is on the head, where airflow direction, 303, come from top air intake modulus, and enters an air intake HEPA filter, 304, that may take out any residual particle down to 0.3 microns, and a carbon dioxide stopper, 305, to take out some organic scents, being alkaline, some germs and of course CO₂. This make this filter heavy, in order to withstand CO₂ polluted environments, it needs to contain about ¼ kg of active material.

Air preparations filter, 306, with medicine applicator for nasal inhalers, may contain an ultrasonic water nebulizer, with all the stuff dissolved in water, where ultrasonic water nebulizer, 307, does humidity control, and may contain fan and batteries, in order to adjust pressure in the exhaust tube, 308, with measurements down on airflow direction, 309.

In exceptionally contaminated environments, the oxygen or compressed air input port, 310, may be used, in parallel by adding a supplementary one-way valve in the middle of the modulus, and this will increase the protection factor to more than −50 dB, but it will add weight and will operate for a limited period of time.

Complementary to its comfort-oriented functions the modulus exhibit some virus and bacteriological protection factor, as estimated in Table 2 below:

| Element | Min. P_{f} | Max P_{f} | Comment |
|---|---|---|---|
| Intake HEPA filter | −10 dB | −20 dB | May stop aerosol |
| | | | particulates attached to |
| | | | watery droplets |
| Carbon dioxide | −10 dB | −30 dB | Exposes the viruses to |
| stopper | | | aggressive alkaline |
| | | | ions |
| Air preparations | −3 dB | −10 dB | Water scents, medicine |
| filter | | | and salt content |
| | | | may react with viruses |
| Total | −23 dB | −60 dB | An average of −20 dB |
| | | | one may count on |

It comes visible that up to now at minimum these two modules are providing a protection of about −50 dB, that is by more than 1,000 times better than the actual mask and PPE wearing, that was proven of having a chance of contracting COVID-19 virus as 1 in 25 to 1 in 100 exposures to infected air spaces.

FIG. 4 shows details on exhaled air cleaning modulus, which is designed to kill viruses in exhaled air, supposing that the bearer is contagious and asymptomatic, as the cheapest cleaning mode, by reducing the emissions directly from the source.

It has a metallic case, 400, in which exhaled air intake tube, 401, sets airflow direction, 402, towards inner case to cool the air in a heat exchanger structure, 403, driving airflow direction in heat exchanger, 404, because reducing its temperature is important exhaust feature because it reduces its buoyancy and its capability of rising up in enclosures as concentrated plume.

To do that, it has a central tube, 405, to drive air in opposite direction towards of an endcap that makes airflow exiting central tube, 409, turn around and spread, entering a desiccator and hydrophilic filter, 406, then a chemical and anti-bacteriological filter, 407, and then a supplementary active charcoal filter; 408, to eliminate mouth bad smell. All along the empty paths being exposed to UV-C light sources, 410. Airflow exiting the heat exchanger, 411, is now at the environment's temperature when air is inside exit tube, 413, maintains airflow direction, 412, when released in the enclosure or into vacuumed drain tube.

Now, this modulus has its designed purpose to protect the society from an infected individual, and brings the protection factors listed in table 3, below:

| Element | Min. P_{f} | Max P_{f} | Comment |
|---|---|---|---|
| desiccator and | −10 dB | −20 dB | May stop aerosol |
| hydrophilic filter | | | particulates attached to |
| | | | watery droplets |
| chemical and anti- | −10 dB | −30 dB | Exposes the viruses to |
| bacteriological filter | | | aggressive chemical |
| | | | and organic materials |
| active charcoal filter | −3 dB | −10 dB | Water scents, medicine |
| | | | and salt content |
| | | | may react with viruses |
| UV-C light paths | −20 dB | −40 dB | Damages structure by |
| | | | 3 eV photon |
| | | | ionization and molecular |
| | | | bound breaking |
| Total | −43 dB | −100 dB | An average of −30 dB one |
| | | | may count on |

FIG. 5 describes how a transparent mask with sense valves, is made.

A transparent mask, 500, is called like that to distinguish from other masks, and as a difference to other masks it has a very soft, low density plastic material to cover the mouth surface, just stiff enough not to collapse at vacuum made during inhalation, but thin enough to absorb as little as possible from the sound emitted by mouth. It has a pair of straps, 501, and air intake sense, 502,

Is given by a connection tube, 503, which contains inside a one-way air valve, 504, aligned accordingly. For comfort and safety purposes a microphone, thermometer, 505, and other bio-medical parameter transducers connected to an electronics box, 506, with Wi-Fi, that may emit a

Wi-Fi signal, 507, complex enough to connect to phone and nearby computers in an ad-hock network. Front of mask visualization, 508, is open but on sides it has connectors for inhaling airflow, 509, and exhaling airflow, 510, that also has a one-way valve for exhalation, 511, before exhalation tube, 512, that allows exhaled airflow in exhaust tube, 513, only. Exhalation sensors (temperature; airflow speed, humidity, CO₂, etc.), 514, are connected to an electronics box, with display, 515, able to communicate via a Wi-Fi signal, 516. In order to allow hassle less hydration, a straw hole, 517, with lid for liquid sipping via a straw. Using protective eyewear is possible to fog, and in order to prevent that, a tube for air to protective glasses, 518, was added on inhalation connector to provide fresh air and keep humidity dew point low enough to keep the visualization surface free from condensate.

The use of mask alone is providing a public protection factor of −10 dB to −20 dB by reducing the spit aerosols range from 2 m down to ¼ m, and accordingly the contaminated surface and volume airspace during exhalation.

An embodiment of the present invention is that airflow is coming from above the head during inhaling, 518, using the semi rigid, inhaling tube, or a hat like fixture, while the exhaled air is sent downwards at the ground level, in order to prevent aerosol droplets to immediately to be inside inhalation range.

Airflow is sent downwards during exhaling, 519, in order to increase relative group protection.

FIG. 5B shows a classical military or professional style, gas mask, 521, mounted on a head, 520, using head bands, 526, which has a dual inhaling filtering cartridge, 523, on the right and a second filtering cartridge, 525 as a surrogate for complete gear, with necessary improvement as an embodiment of the current invention.

Mask has an inlet one-way valve for inhaling, 522, for airflow intake, 524, that is sucked from lower cartridge air intake level, 529, at neck, 531, level instead from recommended air intake position, 527, in order to suck above air, intake upper-level, 528, that is supposed to be cleaner at least by a factor of 2 (−3 dB). A 3 dB is the safety lost by this difference in intake level, 530, from the protection coefficient. This evaluation is done in the assumption that everybody is delivering the exhaled air low at the ankle level for air exhaust, 537.

The mask, 521, is equipped with an exhaling valve, 532, that following the teaching of the present invention is connected to an exhaling tube, 533, driving airflow to exhaled air sterilization and cooling modulus, 534, that delivers cleaner air into sterilized exhaust air tube, 535, with its end at ankle level, 537, setting airflow direction, 536, downwards and backwards towards ground.

After air sterilization, cooling is an important feature, because that dims the exhaled air buoyancy that gives the exhaled plume a lift, and increases the probability that the plume to reach the inhaling level.

The difference in inhaling spot of 1-2 ft. translates in loos of protection factor of 2-3 dB. For a 6 ft. person this procedure of inhaling up and exhaling down, gives a protection factor of about 20 dB. The inhaling filter gives another 10-20 dB, if ordinary filters are used, while the sterilization and cooling canister provides the society with another −30 dB at protection factor that may be added to each person.

In order to clearly understand this calculation, one has to understand that after cooling the exhaled air from 37 C down to 20+C, the exhaled air loses the buoyancy and only diffusion and air currents contributes to its spread. Suppose now one puts down near ground 8 liters of exhaled air, containing a lot of watery droplets (about 10,000/cc according FIG. 14). The diffusion in the absence of air currents evolves in spherical symmetry. In order to reach 2 m at the air inhaling place, it will diffuse in a semi-sphere with radius of 2 m. The volume will be of about 16 m3, and we will ignore the ground effect, that is usually colder and a part of watery droplets may condensate on it, because that depends of the dew point, which is variable in time and with different environments, with different air humidity.

The air dilution becomes 81/16 m3=1/2000=−33 dB. If the inhalation takes place at 1.5 m height, the volume becomes of about 7 m3, and dilution becomes −28 dB, therefore it loses 5 dB.

In the case when one releases the exhaled air at mouth level at a height of 1¾ m, the exhalation plume will be immediately available for inhalation having a concentration between 50% and 10% suffering an attenuation of about −10 dB, therefore, we considered that the protection factor induced by controlling the position of air inhalation, as high as reasonably possible and air exhalation, as low as possible a −20 dB gain in protection factor is obtain, by this behavior feature only. Using filters adds to protection as we have already showed, but using the small, on mask filters as shown in the FIG. 5B instead of canisters on shoulder or up, referred as modules, will trigger another −10 dB reduction in safety factor.

In this surrogate condition, a person may reach a protection factor of about −70 dB that may be good enough, by orders of magnitude of what a personal protective equipment usage today delivers, and that is about −15 dB, and about 1-4% of people exposed get sick.

At a limit of cost and practicality, a N95 or N-100 dust mask with an exhalation valve becomes acceptable if the exhalation valve is connected to a hose, guiding exhaled air into a sterilization canister and from there towards the ground being released at the ankle level. The inconvenience for this mask is the difficulty of speaking inside, and the fact that about 10% of exhalation flow goes in reverse through the mask, as a result of balancing the dynamic pressure drop between one-way valve and mask's filter surface.

FIG. 6 gives details on an enclosure with controlled air flow that is necessary to assure a high degree of safety by default.

Each enclosure utilizes walls or airspace boundaries, 600, that are limiting the airflow and person standing, 601, circulation inside a finite volume of air, where if a person resides for duration of time the exhaled air accumulates increasing its concentration. In normal conditions Exhalation plume without guiding at mouth level, 604, is done, but in pandemic times this has to be prohibited because exhaled air, even though a face mask, that does not stops everything, as shown by FIG. 14, has initial buoyancy rising up on a trajectory of an upwards released exhalation plume, 619, than flowing down, and may be inhaled naturally by another person preset in same airspace.

A protected space has down going airflow, 602, that protects even in the case when exhausted down exhalation plume, 605, rule was breached, pushing everything airborne downwards or parallel with the floor air-stream, 603, with an airflow towards the HVAC return duct, 606.

Airstream aiming downwards, 607, is an efficient protective measure able to provide a protection factor between −10 dB up to −30 dB, because all air goes into return vent of HVAC, 608, from there via an inner tube adaptor to HVAC, 609, return tube of HVAC, 610, where is placed an UV-C light source, 611, just before entering the HVAC unit, 612.

Conditioned air exhaust HVAC, 613, goes via a conditioned air distributed funnel, 614, becoming so called clean, conditioned airflow, 615, aiming downwards.

Radius of exclusion around person, 616, is about ¼ m around the body of a person, where a trajectory of the downwards guided exhalation plume, 617, is supposed to never reach minimum level of inhalation, 618, a height where a kid's nose or an adult bending forward might place the nose, and inhale inside the contaminated plume.

FIG. 7 describes an exhaled air evacuation and sterilization system that is made considering that the easiest is to collect exhaled air only, sterilize and disposition in environment safely, instead of dealing with all the air inside an enclosure. Due to pressure distribution, one has to have a clean air intake, 700, collecting air from outside, and after conditioning, place it in a clean air distribution duct, 701, and blow out via a clean air diffuser, 702, that is providing clean airflow for breathing, 703.

Exhaled airflow collection, 704, is done by driving the sterilized or not, exhaled air, from the mask or sterilization modulus, into a vacuumed drain tube, 705, placed as a floor level collector with lid, 706, that has an end of tube floor level collected airflow, 707. The drain tube has lateral floor level collected airflow, 708, with automatic shutters, opening only when a person is detected nearby and not connected to person's collector exhaled air tube, 709, based on person RFID label, 710, readout by a nearby drain tube RFID reader, 712, placed on person's exhaled air tube connector to drain tube, 711, that communicates to computers via Wi-Fi signals, 713.

The exhaled air goes into a drain air collector sterilization system, 714, has a cyclone separator 715, that retains larger particles into a chemical solution pool on the bottom, illuminated by a UV-C light source, 716, and followed by a pre-filter, 717. After air enters a pre-filter, that retains large dust particles, there is placed a chemical anti-bacteriological filter, 718, and a after that a desiccator and a HEPA filter, 719, cleaning the air before entering in an air turbine, 720, that makes the vacuum, and pushes the air into exhaust tube, 721, from where the exhaust airflow, 722, is pushed safely outside. In spite, some may say, that the collected exhaled air may be flushed outside without any sterilization; we consider that doing so it remains a probability for the virus to survive and spread on unexpected carriers. The sterilization system mentioned above is designed to protect community against unexpected virus propagation paths.

The flow and energy economy for this system is higher, because for a class or bus with 20 seats, occupied by man, the flow is of about 200 l/min compared with a HVAC for the same enclosure that recirculates the entire volume of about 100 m³ in 4 h at a flow rate of 25 m³/h compared with about 12 m³/h needed for exhalation release only. The big advantage of this system is that is taking out from air circuit potentially contaminated air exhaled by a potentially contagious person. In Table 4 below are given the protection factors relative to each element:

| Element | Min. P_{f} | Max P_{f} | Comment |
|---|---|---|---|
| Room airflow structure | −10 dB | −20 dB | Eliminates the |
| | | | potentially dangerous |
| | | | aerosols directly |
| Cyclone filter and UV | −20 dB | −30 dB | Exposes the viruses to |
| | | | aggressive chemical |
| | | | and organic materials |
| chemical and anti- | −10 dB | −20 dB | Exposes the virus to |
| bacteriological filter | | | chemical reactions |
| desiccator and HEPA | −10 dB | −20 dB | Stops watery droplets |
| filter | | | |
| Total | −50 dB | −90 dB | An average of −40 dB |
| | | | one may count on |

It is possible to add a CO₂ retention, alkaline filter, but that might be heavy producing about 20 kg of CaCO₃ in 8 h, but will introduce at least −10 dB in protection factor.

The system will have to be in standby in an institution and enforced any time a contagious person is detected in the area, wear for 2-3 weeks, until everybody is tested and certified healthy, and re-stored to be used at next alert.

FIG. 8 describes a student walking on a protected hallway where, where it is impossible to stay connected at any drain plugs near the wall, therefore a student walking, 801, on a hallway's floor, 800, and wearing a transparent mask, 802, microphone and other transducers, 803, for bio-medical applications is releasing his exhaust near floor, at feet level.

He may use an upper air intake modulus, 804, with or without air preparation modulus, may use loudspeaker and electronics box, 805, to conveniently amplify his voice while speaking inside the mask, or remotely connecting.

Exhaled air tube, 806, connects mask's exit to exhaled air sterilization modulus, 807, that may be placed on the leg also, from where sterilized exhaled air flow, 808, creates a sterilized exhaled air plume, 809, integrating in airflow near floor, 810, which is going into HVAC return vent, 811. From the vent air is passing through HVAC horizontal return duct, 812, into HVAC vertical return duct, 814, where is placed an UV-C light source, 815.

The HVAC unit, 816, may be set in cooling or heating mode, where air is going through the HVAC conditioned air ceiling duct, 817, to conditioned air ceiling diffuser, 818, that forms it as a conditioned air down flow, 819, inhaled by student as clean, virus free air.

Because the exhaled air is released low, and the COVID-19 virus propagates through skin too, students have to cover all skin surfaces by wearing pants and socks, 820.

In the case a student fails to wear a mask, he might still have a protection factor higher than −15 dB assured by the building airflow. If the hallway is exposed to wind and turbulent air currents, the protection factor may be as low as −10 dB, still protecting student as equivalent to wearing an N95 mask. Policies of wearing the full system have to be enforced to have all systems in synergy.

FIG. 9 shows students traveling inside a protected school bus, sitting in a school bus sector, 900, provided with clean air input, 901, that makes a clean air down flow, 902, reaching upper air inhalation module, 903, sitting up on a semi-rigid connecting tube, 904.

Student on the aisle, 905, and student at the window, 906, are wearing transparent face mask, 907, connected to inhalation air secondary filter module 908, visible on student on aisle, and in part, an inhalation air secondary filter module on student at window, 909, connected via a flexible tube to transparent face mask at student at the window, 910.

Each face mask is connected to an inhaling air tube, 911, and to an exhaling air tube, 912, while only an inhaling air tube at student at the window, 913, is visible.

Behind them there is a girl student wearing long socks, 914, while student at the window backpack, 916, is on seat.

The exhaled air, goes first through an exhaling air sterilizer modulus, 915, that is exhausted as disposable, into a sterilized exhaled air exhaust tube, 917, which has an RFID label, 918, to be read when inserted into sterilized exhaled air tube connector, 919, that has a tube connector box to drain with lid, 920, and an RFID reader, 922, that identifies the student, and transmits the status via Wi-Fi signal, 921, to nearby computers. A connecting tube to the drain tube, 923, is placed at student convenience, to make the connecting to drain tube, 924, easier and reliable.

Drain tube end lid, 925, and lateral lid on vacuumed drain tube, 929 have automatic shutter/opener at presence detection, 930, and open instantly when detecting a nearby presence that is not connected with its exhaust to drain tube, and performs airflow collection on the floor, 926, and drain airflow to exhaust system, 927, where is cleaned up again and disposed into environment. With all this, wearing pants with socks, 928, it is recommended in order to avoid COVID particulates entering in direct contact with the skin, and diffusing through.

FIG. 10 shows a school bus driver watching kids walking on street through his bus windshield, 1002.

Front school bus windshield middle ridge, 1000, separates front windshield and on the left is a school bus driver, 1001, wearing eye glasses, 1003, and having an air flow in driver's space, 1004, coming clean from outside. He wears a transparent face mask, 1005, that allows him exhale into a tube connector at mask one-way valve, 1006, followed by an exhaling tube, 1007, which connects the mask to exhaled air sterilization modulus, 1008, that cleans his exhalation air and sends it into a sterilized exhaled air connection tube, 1009, with the connector placed on bus dashboard, 1010, where it has a sterilized exhaled air connector to drain tube, 1011, with an RFID label, 1012, read by a nearby RFID reader, 1017, communicating via a Wi-Fi signal, 1013, with a computer system, and letting the bus driver know when it is not connected his airflow to drain tube, 1015, in order to automatically open the shutter and maintain airflow direction, 1016, through drain tube connector and lid, 1018.

He was watching the kids on the street, because in spite they were equipped with a complete system, as required to travel with the bus, they committed various system protection breaches endangering each other.

A sterilized exhaust plume is released correctly on the street level by middle girl, 1019.

Left side girl, 1020, took off her mask, and has inhaling tube, 1026, not connected to mask, nor her exhaling tube, and releases directly in air a plume of exhaled gas, 1028, which may be potentially contaminated.

The student on the right has air flow inhaling intake direction, 1021, right, towards air inhaling modulus, 1022, from there in its connecting tube, 1023, via inhaling air filtration unit, 1024, into face mask 1027, but disconnected her exhaling tube, and released her exhale plume, 1033, of exhaled air at mouth level through her mask, straight in the face of the middle student.

The middle student is wearing her transparent mask, 1025, correctly, having the tube connecting transparent mask to sterilization modulus, 1029. Exhaled air is cleaned in her sterilization of exhaled air modulus, 1030, and sent via tube exiting the sterilized exhaled air at street level, 1031, producing a plume of sterilized exhaled air released at street level, 1032.

Even if the student on the right was contagious, the middle student survived the aggression, remaining uncontaminated, because her breathing level was up, above the plume, and has inhalation filters working, but the left side student coming from behind may have inhale air inside that plume, taking about 30% of the right student exhalation, and she get infected. There is a risk on middle student to have exhalation droplets from the plume condensate on her face, as she is not wearing a face shield. The risk figures for the students are of about −12 dB for the middle student in spite her protection of over −30 dB, due to face exposure to warm aerosolized plume, that shortcut the protection, and about −3 dB for the right student. The left student is equipped well, with pants and is protected against the clean exhaled air from the middle student, having a −30 dB protection at legs skin diffusion level, but remains fully exposed at respiratory level, by not wearing the protective system and walking straight in the contrail of the right student. In reality, appears what is called function of luck, because in many cases of breaching the protections nothing bad happens, but this appearance of luck is in fact a probability as a contagious person to be involved, that is given by the ratio between estimated contagious people at large to the effective population involved in contacts in that area. This multi-parameter function is complex and mainly unknown, but that acts as a supplementary protection factor with values in the domain between −10 to −30 dB, and that is what we rely when we say that temporary breaching some protection stages it is still safe to operate, and the problem is not to breach them all to remain inside luck realm.

FIG. 11—Student working on computer inside a protected space has also to wear the protective equipment if the space is for public access. When a student is working at computer in a protected environment, 1100, he uses fresh, clean air coming from above vent, 1101, that has a clean airflow direction, 1102, towards his inhalation top modulus, 1103, that may either stay as a hat, on his head or use a semi-rigid tube for connection, 1104, with supplementary modulus that is preparing respiratory air, connected to his face mask air input, 1105. He may be contagious or he may be healthy, he might not know if he is asymptomatic, but in any case he wears a face mask that has an exhaled air exit connector, 1106, to accommodate exhaled air connection tube, 1107, that drives the air to an exhaled air sterilizer modulus, 1108, and from there, via a sterilized exhaled air exhaust tube, 1109, into a vacuumed drain tube, 1116.

In order to make the process easier, the connection of exit tube to drain pipe, 1110, is equipped with a RFID label, 1111, read by an RFID reader, 1114, which emits a Wi-Fi signal, 1112, when connection box with lid, 1113, that connects student's exhaust tube to tube to drain tube, 1115, has the switch status changed, informing a local computer.

At the end of drain tube collected airflow direction, 1117, is set by drain tube's suction, and so is for lateral drain tube opening airflow direction, 1118, which penetrates through lateral opening with lid of drain tube, 1119, and from there drain tube airflow direction to sterilization unit, 1120.

FIG. 12 shows another instance for people in a protected bus, train, airplane, meeting room or amphitheater, that have to use same system in order to safely load the space up to maximum capacity safely.

In all spaces such as a bus, train, plane, conference hall frame, 1200, upper vent for introducing clean air, 1201, where clean airflow direction, 1202, is towards each seat, 1203.

In the area, there is a person sleeping, 1204, a person sitting, 1205, having upper air inhalation modulus, 1206, sitting on an upper modulus connection tube, 1207, connected to respiratory air filter modulus, 1208, and further having an exit to connection tube to mask, 1209.

All of them wear transparent mask, 1210, and is called “transparent”, because is made by low density polyethylene or other transparent, low density carbon sheet, has no filtering capabilities, but sense valves, and basically blocks the direct spit range, by guiding intake and exhaust via exhaled air connection tube from mask exhaust valve to sterilization modulus, 1211.

Exhaled air sterilization modulus, 1212, is not really needed when one is connected to a drain tube, but when mobile and connection becomes is impossible, it is better to use it with sterilization modulus exhausts tube, 1213, lose, but near the ground to dispose exhaled air safely.

In order to know when this tube is connected to vacuumed drain tube, 1220, and when it is not, a RFID label, 1214, that is read by an RFID reader, 1218, placed nearby on connection port, 1216, that detects if seat's connection tube on drain tube, 1219, is plugged in connection tube with lid, 1217, and using Wi-Fi signals, 1215, transmits the information to local computer.

Drain tube also has lateral port with lid, 1221, to collect airflow and direct, 1222, it working together with end of drain tube, that sets airflow direction, 1223 towards drain tube airflow to sterilization unit, 1224, where is cleaned again, before being dumped in the environment. A simple solution is to dump it directly in open air, but that is a hazardous irresponsible action, because is dumping a bio-hazardous agent able to survive and latter transmit and infect.

FIG. 13 shows students inside a protected classroom, as a way of performing classroom activities in person, where there is shown a classroom selection, 1300, only to present a way this may be done safe, during high contagiousness times, for a short period under 1 mo. for everybody in a designated infected area, to terminate and eliminate infection, and buy time such all susceptible persons to be accurately tested, or it may become a way of life for uneducated populations, preserving COVID-19 propagation, based on social and religious beliefs .

Inside selected area, there are only students, one right side student, 1301, one middle row back student, 1302, one middle row front student, 1303, a left row back student, 1304, a left row front student, 1305, placed under ceiling diffuser for HVAC, 1312, setting conditioned airflow direction, 1313, coming clean and in part fresh from above straight in students' upper inhalation modulus, 1314, sitting on a semi-rigid connection tube to inhalation filter modulus, 1315, which drives the intake air into inhalation filter modulus, 1316, air flow direction in the HVAC return, 1306, and from there inside transparent mask, 1317, through connection tube to transparent mask intake one-way valve, 1318, making student breathe easily. The transparent mask also has an exhalation connection tube, 1319, guiding the exhaled air, possibly infected, into a sterilization of exhaled air modulus, 1320, and from there into vacuumed drain tube, 1322, via a connection tube carrying sterilized exhaled air into drain tube, 1321. The vacuumed drain tube has a Left side drain tube end, 1323, and a right side drain tube end, 1324, being possible to be made by a single flexible tube, made a U shape on floor, connected in the middle to sterilization exhaust unit.

This floor placed vacuumed tube, works redundantly with building's HVAC system which collects floor air into HVAC return vent, 1307, from where it passes into HVAC vertical duct, 1308, where it is sterilized using a UV-C light source,1309, placed before HVAC unit, 1310, which is conditioning the air and returns it via a HVAC horizontal ceiling duct, 1311, into class.

The HVAC unit may recirculate or even refresh air in order to bring in class air as fresh as possible, having the main purpose of it to collect any exhaled air from mobile people as teacher or students at the board, from spreading into the entire airspace.

FIG. 14 is given as a courtesy for the reader, and shows a chart of exhaled water droplet distribution vs. filter retention capabilities, because the information contained here seems unknown to top CDC scientists and leaders, and based on this ignorance a lot of misinformation circulated as reference to COVID-19 average dimension of 100 nm, 1401, with a reasonable droplet size scale, 1402, ranging from 0.1-100 microns. On left ordinate axis is represented the number of particles per cc (cubic centimeters) as function of particle magnitude, 1403, and also particle filter 100% pass upper limit, 1404, is conveniently placed, on top, while particle filter 0% pass=100% stopping power, lower limit, 1405, is placed on the opposite side. Time airborne droplets float in air scale, 1406, where the droplets have 1.5 g/cc density, is there to show how long a certain type of droplets may be resident in a closed room atmosphere and may accumulate. In the upper right corner it is presented the case of a person that exhales and sets in air droplets that may or may not contain viruses inside, where their concentration is given by a color code, 1407, and applies for the virus inside an air exhalation too. Exhaled air-jet change of direction due to mouth cover, 1408, and makes it turn downwards, while exhalation plume without any face cover, 1409, may go as far as 8 ft (2.4 m), and is also influenced by wind's direction, 1410, in correlation with the orientation of the head of a contagious person, 1411.

In order to understand how a protective measure works, one must first understand the dimensional distribution of airborne droplets while coughing openly, 1412, then, what happens when coughing is performed with retention in a new N95 mask, first shout, 1413, where conversion of large droplets into small droplets released by mask by atomization, 1414, takes place, making the things more complex due to extending the airborne time of droplets, with increased accumulation. Dimensional distribution of droplets released through a paper-towel filter, 1415, is a little bit larger, but atomization process is not as strong and the release of droplets spectrum through is smaller than that for a N95 mask (without exhaust valve); 1416. But overall, the results are about the same. In order to understand the uselessness of a N95 mask or filter, one has to consider the dimensional distribution of droplets during an open exhalation, 1418, versus normal breathing and air exhalation through a mask, 1417 and see that at best, when new it is a 50% reduction only, while the liquid effluents accumulated in the mask are further released, in the dressing/undressing room and storage room where most of contaminations take place.

The airborne time was approximated with the time it takes a droplet to free-fall 1 m (3.3 ft) based on Stokes force, represented by the solid line, 1419, and over for clarification is given in percent the paper towel filter retention curve, 1421, and N95 filter retention curve, 1422, where one may see that none of them really stops under micron particles, nor liquid effluents.

The details in the figure allow one to understand that a filter does not stop all particles; solid fiber filters behave differently for solid particulates and for liquid effluents where micro-nano-fluidics come into play. Readers have to understand also that a volume that was filtered once through a filter gets little to no change in particle distribution if it is filtered again through same type of filter. A filter produces only attenuation in particles' density of about 20 dB, and skin behaves as a porous membrane in relation to virus producing an attenuation of about 20 dB up to 30 dB due to sweat and other organic odors that interact with viruses. The virus transmits as aerosols more efficiently than as diffusing through skin, but skin hygiene is important factor that affects skin attenuation factor, and washing is affecting protection in both directions.

It has to be understood from the very beginning that COVID-19 propagates via a plurality of transmission processes, as 90% is airborne respiratory transmission, 5-8% is transmission via surface and hands role in helping the virus go through sensitive places of the body and mouth, eye liquids, and up to 2% direct skin through transmission, therefore in order to stay safe all these transmission paths have to be denied, by a plurality of common sense measures, as cover the surfaces, wash hands, keep distance, and FIG. 14 shows just that. The invention focuses on aerosol transmission path, because this is the most complicated and less understood part by public up to medical organizations as CDC, WHO, etc., and clarifying these measures may effectively stop the pandemic without registering economic losses or disruptions, but requires a smart community, and that is hard to find, therefore wearable electronics and firm policies have to be introduced to counter for this type of failure.

FIG. 14 presented data was used to evaluate the protection factor needed in order to make a person stay in the space of a COVID-19 contagious person safe without using a full body protection equipment with own air source.

We have learned that a person exhales about 10,000 watery droplets per cubic centimeter, and exhales about 10 liters per minute.

In order to be safe a person nearby that inhales 10 liters must find less than 1 droplet, by concentration dilution or by elimination mechanisms.

A simple math says that a person exhausted 10⁸ particles/min which we have to reduce at 1 particle/min for a similar 10 l air inhalation and that simply that require −80 dB in protection factor. At this concentration, keeping in mind that there is a corpuscular behavior, one volume may content none, and other may contain a few, therefore another 2 orders of magnitude are needed and that drives roundly to about −100 dB as shielding desired value, and if one reach that it is good enough.

Another case is when a contagious person is confined in a 10 m³ space for 10 h, exhaling 10 l/min.

The total particle concentration will be: 10,000 [particles/cm³]×10[l/min]×60[min]×10[h]/10[m³]=6 10⁹[part/m³]. If the receiver person inhales about 10 l/min it will get 6 10⁷[part].

This is about the same as in previous case, where −80 dB protection factor upgraded at −100 dB may do the job. The reason we used dB is because it is an attenuation, the curves are exponentials, with an asymptotic trend to zero, until in corpuscular, low concentration domain, where statistics randomness takes control. At −100 dB, we may say that we may inhale 99 min air with no particle and 1 min we may inhale 1 particle. The average safe time in a contaminated area is about 1 h. If we need more safety, we need to add more protective equipment and procedures, in order to reach −120 dB, and that was the goal.

The problem is that filtration systems can get this figure with the price of high pressure drop and large volumes that render them as difficult to bear as individual protective equipment with own air, and the only way to make a safe, cheap and easy to bear is to distribute the protection factor between the contagious person, enclosure, and the healthy person, not knowing who is contagious and who is healthy, until symptoms or test whichever occurs first will take out the contagious person.

We want to state clearly that without testing, quarantine and medical action the equipment have to be used continuously, or for a longer period of 2-3 months until the pandemic decays and dies by itself. If one leader opts for no testing, no knowledge, it may work well if it uses the patent for a period longer than 4 months at large scale, and that may reduce even the need for vaccination against common flu, but that is what we do not recommend, as body self-immunity is an add-on to protection factor brought by this patent.

### EXAMPLES OF THE INVENTION

The current idea was to create a redundant, synergistically, individual and collective protection system that to be used by the entire population of a region where SARS infection started, in order to completely eliminate it in the shortest time possible, or to be used inside an infected community that is poised to remain infected, assuring the safety of users only, and leaving that infected specimens to be eliminated by nature in time.

Wearing this equipment, people may enjoy the proximity of others without any other visible separators, being free to fail temporarily various modules of protection without having a drastic catastrophic failure ending up with an infection.

Of course, if the method and equipment is not followed properly, the protection factor given may be entirely lost, but that requires a set of simultaneous failures, or a type of failure as presented in FIG. 10 in the condition where all negative odds are met, as presence of sick person, sick person contaminated aerosol release, immediately after exhaling, at high level, healthy person presence and inhalation in the contaminated plume, without using the inhalation modules, and failure in the enclosure air conditioning systems or lack of those and the presence of a preferential air direction. Adding all these probabilities together, we get a very small number, but it is not absolute zero.

The present patent is not intended to drastically change how parties and interactions are performed, but compared to actual parties where everybody in that room is contaminated with the spit droplets of everybody there, and is breathing micron size watery droplets exhaled, socializing activities will take place in a sterile environment where the participants exhaled air is first sanitized, purified and then recycled, or exhausted and breathing air will be provided from fresh, clean outside air.

For more safety purposes, the protection system may acquire bio-medical parameters, and analyze them for advance detection of any little change in health, or prevent long duration failures to properly use the system.

As examples of applications depicted in FIG. 1 that shown the synergy between individual and enclosure protection system, developed based on rule to inhale air up and exhale it down, while cleaning it all over where it is reasonably possible.

Another example is provided in FIG. 8 that deals with the case of students moving on hallways, where the usage of an extra drain system is not possible, but a high level of safety may be assured.

FIG. 9 shows what may happen on a school bus and how the protection method is applied to assure ultra-high safety.

FIG. 10 is another example of application and also analyzes the potential failures of usage in open spaces as streets or yards.

FIG. 11 stress the case that inside protected enclosures, no matter if a person is alone or not, the same procedure is applied because exhaled air may accumulate and may not be absolutely clean and free of any virus or germ.

FIG. 12 details the fact that sleeping or being awake, the system and method have to be fully used in order to stay safe and sound.

FIG. 13 shows an application in class for in person teaching at schools and colleges where minimal modifications to HVAC system have to be made, plus the installation of a modular, mobile and flexible drain system to deal with students exhaled air and bad mouth odors.

This may be also applied to hospital beds, emergency tents or in any place where a bio-agent emitter is present and its insulation is desired, for example, in a protective equipment (PPE) dressing and undressing room, where the agent is intentionally crossing the curtains, to gradually separate from the contaminated PPE, which in this process is partially sterilized and stored.

The system is modular, flexible, computer controlled, for quality assurance, easy to implement entirely or in part, after analyzing what is reasonably achievable and desirable, but for sure is more than a mask, social distancing and “wash hands” that produces a maximum −20 dB protection factor, and 1 infection to about 100 exposures, that is far from being acceptable in a reasonable society with respect for human life versus money, and is designed to save big money at the society level, because a healthy economy cannot be achieved without a healthy society.

1. A method of holistic protection and virus transmission suppression , that uses the synergy between enclosure infrastructure and individual protective systems that:
a. Relies on keeping clean safe air to breathe above, the wrist and pushing the exhaled air, potentially contaminated down at the foot level;
b. Is using a modified building HVAC system that:
i. has the clean air coming from ceiling and the return air being collected near the flow;
ii. Has a UV-C sterilization unit embedded into the return duct
iii. Uses chemical and anti-bacteriological filters to stop and kill viruses and germs;
c. Uses an additional vacuumed drain system deployed on the floor level that:
i. Directly collects the exhaled air from people via a exhaled air tube connected to the drain tube
ii. Collects exhaled air near floor by the mobile people that cannot plug their exhaled air tube into a fixed drain tube connector;
iii. Uses RFID technology to identify who is in proximity and if it is connected or not and transmits it by Wi-Fi to a computer;
d. Relies on an individual modular protection system comprising:
i. Top air intake modulus containing:
I. Primary solid particulate pre-filter protection grid;
II. Coarse hydrophobic filter;
III. Chemical and sterilization filter ;
IV. Hydrophilic filter-desiccator;
V. UV reflective chamber with bio-active wall;
VI. UV-C light source;
VII. Fan and batteries with electronic control;
ii. Inhalation air preparation modulus, containing inside:
I. Intake HEPA filter;
II. Carbon Dioxide stopper;
III. Air preparation filter with medicine applicator for nasal inhalers;
IV. Ultrasonic water nebulizer, humidity control, fan and batteries;
V. Compressed air or oxygen port;
iii. Transparent mask with sense valves comprising:
I. Transparent mask;
II. Air intake connection tube with one-way air valve;
III. Straw hole with lid for liquid sipping via a straw;
IV. Exhaling airflow, connection tube with one-way air valve;
V. Electronics box, with display and Wi-Fi Communication;
VI. Tube for air to protective glasses;
iv. Exhaled air cleaning modulus, containing inside:
I. Desiccator and hydrophilic filter;
II. Chemical and anti-bacteriological filter;
III. Supplementary active charcoal filter;
IV. UV-C light sources;
V. Heat exchanger
e. Has a portable system of control and data acquisition for quality assurance, and communication comprising:
i. Electronics box—Wi-Fi;
ii. Dynamic pressure control loops for inhalation and exhalation;
iii. Microphone, thermometer;
iv. Exhalation sensors (temperature; airflow speed, humidity, CO₂, etc.);
v. RFID labels on exhaust tube;
vi. Direct display on mask of check parameters.
f. Has a fixed system, at enclosure level to check presence and protection equipment operation status;

2. A method of holistic protection and VIRUS transmission suppression according claim 1 that is modular, distributed and has to be used by the entire community simultaneously for a short period of time, immediately after a pandemic source was identified, until the threat is removed;

3. A method of holistic protection and VIRUS transmission suppression according claim 1 that has its quality assurance procedures that have to be enforced and aimed for maximum protection in order to leave room for reasonable failures that may occur, and fail safe;

4. A method of holistic protection and VIRUS transmission suppression according claim 1 that relies on intense cooperation between people, wearable technology and enclosure technology in order to produce synergy;

5. A method of holistic protection and VIRUS transmission suppression according claim 1 that has independent protective structures, where the total failure has a small probability, being mainly the result of a conscientious self-sabotage action, that will be immediately detected by the monitoring systems and corrective action triggered;

6. A method of holistic protection and VIRUS transmission suppression according claim 1 that is may be used for open space protection too, by simply using the mask and the two air-tubes correctly placed.

7. A method of holistic protection and VIRUS transmission suppression according claim 1 that is requiring a downwards air flow imposed inside enclosures in order to assure protection.

8. A method of holistic protection and VIRUS transmission suppression according claim 1 that requires exhaled air sterilization as a bio-hazard before being dumped in environment or recirculated.

9. A method of holistic protection and VIRUS transmission suppression according claim 1 that considers that there is no absolute protection and favors complementary measures to stop virus propagation, as covering exposed skin, tests and quarantines, etc. in order to eliminate the community hazard in short time, keeping everybody safe and not interrupting or disturb their activities.

10. A protection system that comprises:
a. Positional protection achieved by:
i. Inhaling air from a top intake placed on hat or a semi-rigid tube
ii. Exhaling air as low as possible at foot level by an exhaust tube;
iii. Cooling the exhaled air before exhausting to dim its buoyancy;
iv. Exhausting exhaled air into a drain tube for outside disposition;
b. Enclosure protection made of:
i. Modified building HVAC air flow with air UV and filter sterilization;
ii. Supplementary drain tube deployed on floor, to collect and dispose safely out the exhaled air mainly, only after sterilizing it;
c. Personal protection of wearer individual made of:
i. Top air intake modulus containing:
I. Primary solid particulate pre-filter protection grid;
II. Coarse hydrophobic filter;
III. Chemical and sterilization filter ;
IV. Hydrophilic filter-desiccator;
V. UV reflective chamber with bio-active wall;
VI. UV-C light source;
VII. Fan and batteries with electronic control;
ii. Inhalation air preparation modulus, containing inside:
I. Intake HEPA filter;
II. Carbon Dioxide stopper;
III. Air preparation filter with medicine applicator for nasal inhalers;
IV. Ultrasonic water nebulizer, humidity control, fan and batteries;
V. Compressed air or oxygen port;
iii. Transparent mask with sense valves comprising:
I. Transparent mask;
II. Air intake connection tube with one-way air valve;
III. Straw hole with lid for liquid sipping via a straw;
IV. Exhaling airflow, connection tube with one-way air valve;
V. Electronics box, with display and Wi-Fi Communication;
VI. Tube for air to protective glasses;
d. Community protection by individual made of:
i. Exhaled air cleaning modulus, containing inside:
I. Desiccator and hydrophilic filter;
II. Chemical and anti-bacteriological filter;
III. Supplementary active charcoal filter;
IV. UV-C light sources;
V. Heat exchanger
e. Auxiliary electronics for communication, comfort, quality assurance and health made of:
i. Electronics box—Wi-Fi;
ii. Dynamic pressure control loops for inhalation and exhalation;
iii. Microphone, thermometer;
iv. Exhalation sensors (temperature; airflow speed, humidity, CO2, etc.);
v. RFID labels on exhaust tube;
vi. Direct display on mask of check parameters;
vii. A distributed system, working at enclosure level to check presence and protection equipment operation status

11. A protection system according claim 10, where the air is inhaled high and exhaled low, that works in synergy with enclosure air systems that brings fresh clean air on top and takes out air on the floor level;

12. A protection system according claim 10, where the mask is transparent for facial gesture identification and has one-way valves and straw hole with transparent lid;

13. A protection system according claim 10, where an electronic system is controlling the pressure at mouth level, inside the mask and drives the fans to make pressure positive when inhaling and negative when exhaling;

14. A protection system according claim 10, where the respiratory air preparation modulus have a port that may be used to supply compressed air from a bottle, or oxygen at demand;

15. A protection system according claim 10, where electronics is used to provide better acoustic local communication or remote via Wi-Fi and internet communication;

16. A protection system according claim 10, where a bio-medical parameter data acquisition system distributed in individual protective system is used to monitor and display the health status of user.

17. A protection system according claim 10, where the components may be added or bypassed according to needs and the hazard level estimated to encounter.

18. A protection system according claim 10, where the completeness and functionality of the system is electronically controlled and remotely transmitted into a computer network.

19. A protection system according claim 10, applying redundant protective modules, in order to maximize protection and completely suppress any virus propagation, intended to be used simultaneously by the entire community for a short period of time, immediately after a pandemic source is detected.

20. A protection system according claim 10, which uses the fact the virus is traveling in watery droplets, able to locally dissolve and react with salts and other chemicals that kill the virus, working in synergy with UV-C light, and then seals the virus remains after water evaporates and chemical re-crystalizes
